(19) United States
(12) Patent Application Publication
(10) Pub. No.: US 2022/0332673 A1
(43) Pub. Date: Oct. 20, 2022

(54) CATALYTIC DEPOLYMERIZATION OF LIGNIN TO HIGH VALUE HYDROCARBONS

(71) Applicant: University of Florida Research Foundation, Inc., Gainesville, FL (US)

(72) Inventors: Zhaohui TONG, Gainesville, FL (US); Hanxi BAO, Gainesville, FL (US); Lin Zhang, Gainesville, FL (US)

(21) Appl. No.: 17/621,043

(22) PCT Filed: Jun. 16, 2020
(86) PCT No.: PCT/US2020/037920

§ 371 Date: Dec. 20, 2021

Related U.S. Application Data
(60) Provisional application No. 62/866,730, filed on Jun. 26, 2019.

(51) Int. Cl. C07C 67/333; C07C 41/01; B01J 27/053

(52) U.S. Cl. CPC C07C 67/333; C07C 41/01; B01J 27/053

(57) ABSTRACT

The present disclosure provides for methods for depolymerizing lignin to produce other useful products. For example, low molecular weight aromatic and aliphatic hydrocarbons (e.g., hydrocarbons having 8 to 20 carbon atoms (C8 to C20 hydrocarbons)) as well as oil products can be produced using methods of the present disclosure. The method can include treatment of the lignin using a catalyst composition, where the catalyst composition comprises a persulfate salt and a transition metal catalyst.

### CLAIM OF PRIORITY TO RELATED APPLICATION

This application claims priority to co-pending U.S. provisional application entitled “CATALYTIC DEPOLYMERIZATION OF LIGNIN TO HIGH VALUE HYDROCARBONS” having Ser. No. 62/866,730 filed on Jun. 26, 2019, which is entirely incorporated herein by reference.

### FEDERAL FUNDING

This invention was made with government support under DE-PI0000031 awarded by The United States Department of Energy. This invention was made with government support under 2017-68005-26807 awarded by the United States Department of Agriculture National Institute of Food and Agriculture. The government has certain rights in the invention.

### BACKGROUND

Lignin is the second most abundant polymer after cellulose in nature. It is reclaimed as the major byproduct from pulp and paper and sugar-platform biorefinery industries. It has been largely used as-cost energy source through incineration despite its potential high value as a source of aromatics. Only 2% of lignin has directly been used as commercial products due to its characteristics.

### SUMMARY

The present disclosure provide for methods of depolymerizing lignin. In an aspect, a method for depolymerizing lignin comprising: providing a first composition comprising a lignin and a catalyst composition, wherein the catalyst composition comprises a salt of S₂O₈²⁻ and a transition metal compound; and depolymerizing at least a portion of the lignin to provide one or more of a low molecular weight aromatic monomer, an aliphatic hydrocarbon, or a combination thereof. For example, the salt of S₂O₈²⁻ can include Na₂S₂O₈, K₂S₂O₈ or a combination thereof. The transition metal compound can be an iron transition compound.

In another aspect, the present disclosure provides for a method for depolymerizing lignin comprising: providing a first composition comprising a lignin and a catalyst composition, wherein the catalyst composition comprises a salt of S₂O₈²⁻ and a transition metal compound; and depolymerizing at least a portion of the lignin at a temperature of about 130 to 140° C. to yield an oil product.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 illustrates the GC-MS spectra of reaction medium from the model compound study. Mass spectra confirm the conversion of the model compound ((1R,2S)-1-(3,4-Dimethoxyphenyl)-2-(2-methoxyphenoxy)-1,3-propanediol to veratraldehyde over 60-min.

FIG. 2 illustrates a plot of predicted conversion rate versus observed value

FIG. 3 illustrates contour plots and response surface diagrams between two parameters. From top to bottom, (A) reaction time and persulfate loading at the center level of temperature, (B) temperature and persulfate loading at the center level of reaction time, (C) temperature and reaction time at the center level of persulfate loading.

FIG. 4 illustrates reaction barriers (free energy in kcal/mol) for .OH radical to break chemical bonds of 1-(3,4-dimethoxyphenyl)-2-(2-methoxyphenoxy)-1,3-Propanediol at M062X/6-311G** level of theory.

FIG. 5 illustrates proposed mechanism and the key steps at M062X/6-311G** level of theory (R₁═CH₃, R₂═H; Gibbs free energies in kcal/mol).

FIG. 6 illustrates the yields of depolymerization products of organosolv lignin, organosolv lignin with phenol, and alkali lignin (Reaction condition: 80° C., 1 atm, reaction time 24 hrs).

FIG. 7 illustrate the molecular weight of two types of lignin depolymerized products at different reaction time (80° C., 1 atm).

FIG. 8 illustrates gel permeation chromatograms of depolymerized products from organosolv lignin

FIG. 9 illustrates GC-MS spectrum of lignin oil products (80° C., 24 hrs, 1 atm): 1) Organosolv lignin 2) Alkali lignin. Mass spectra confirm the presence of a multitude of potentially value-adding phenolics.

FIG. 10 illustrates heteronuclear single quantum coherence (HSQC) 2D nuclear magnetic resonance (NMR) spectra of phenolic oil produced from depolymerization of organosolv and alkali lignin.

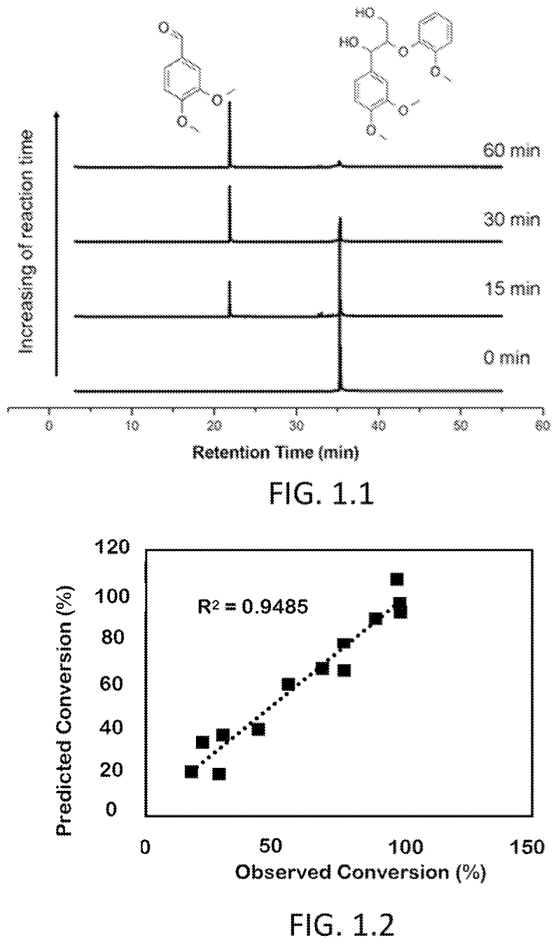

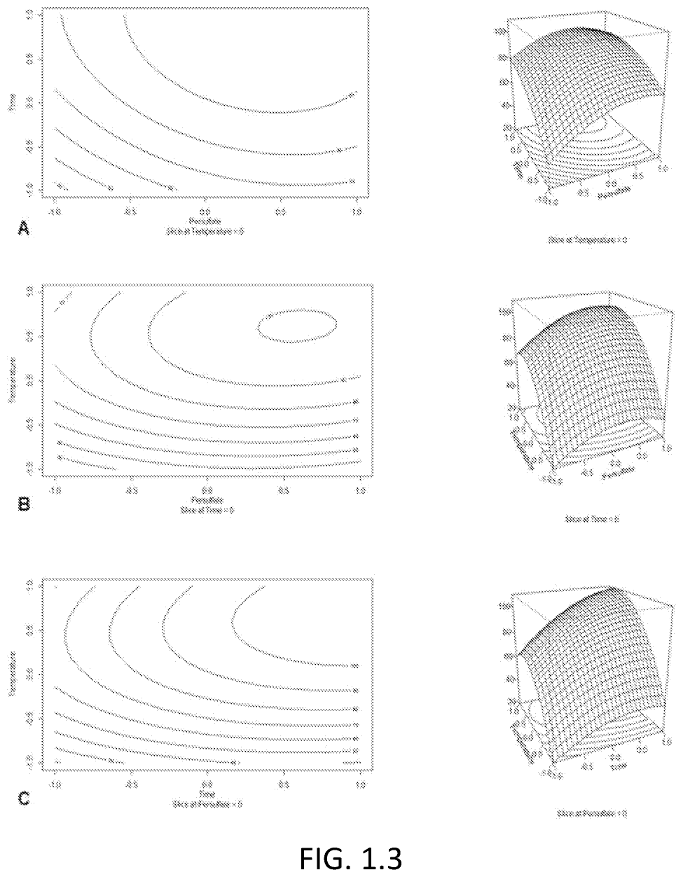

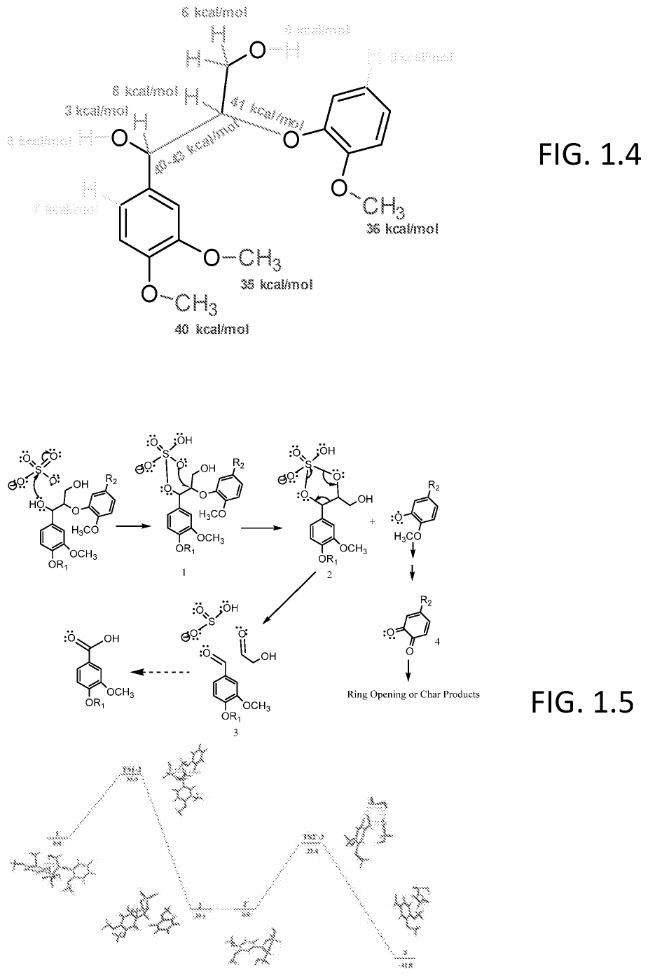

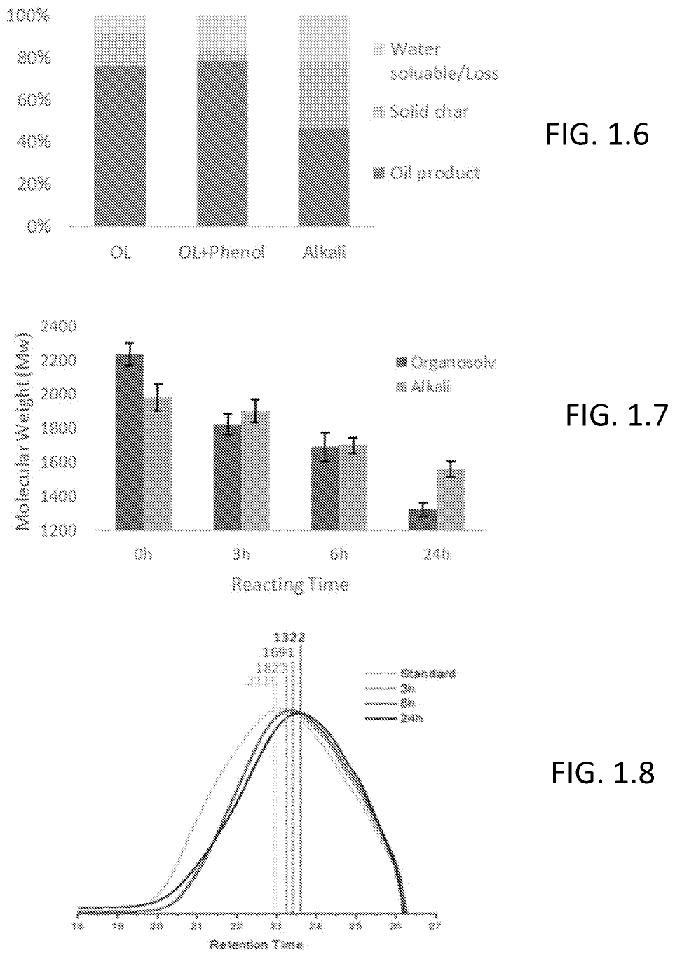

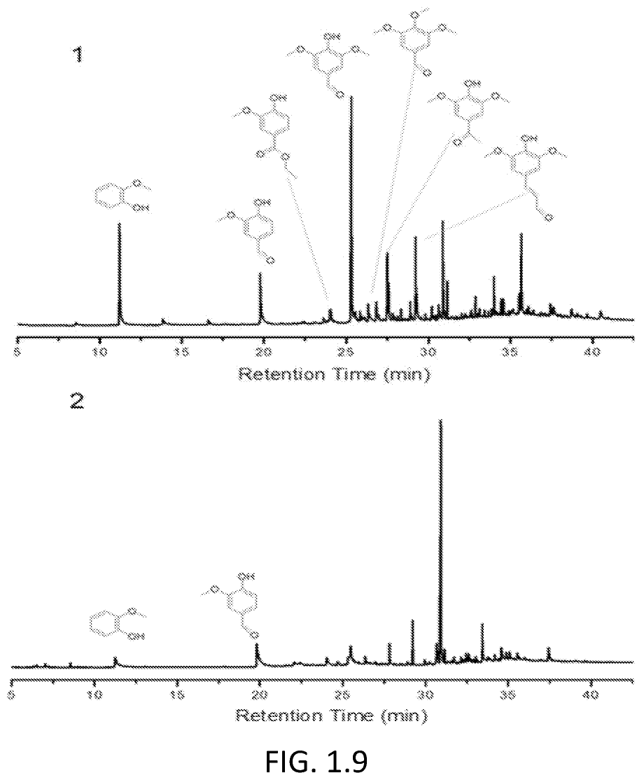

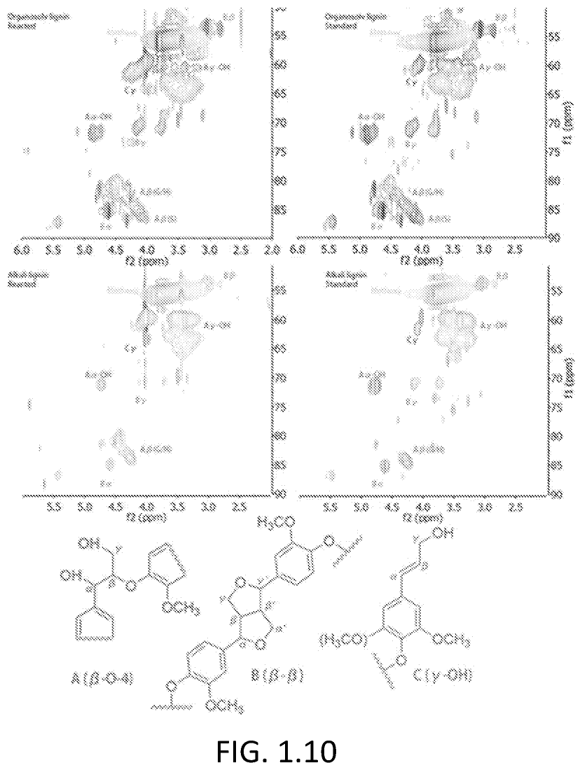

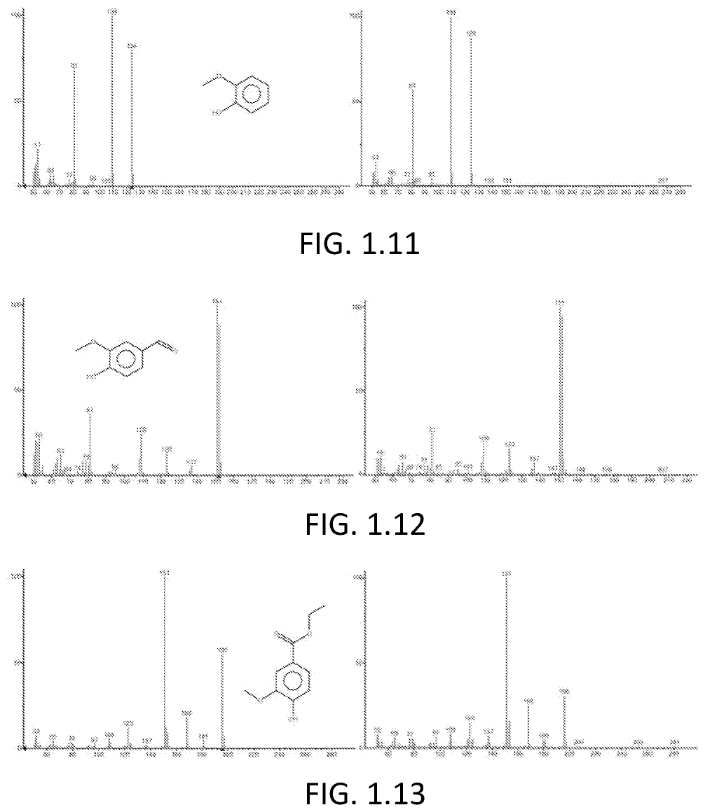

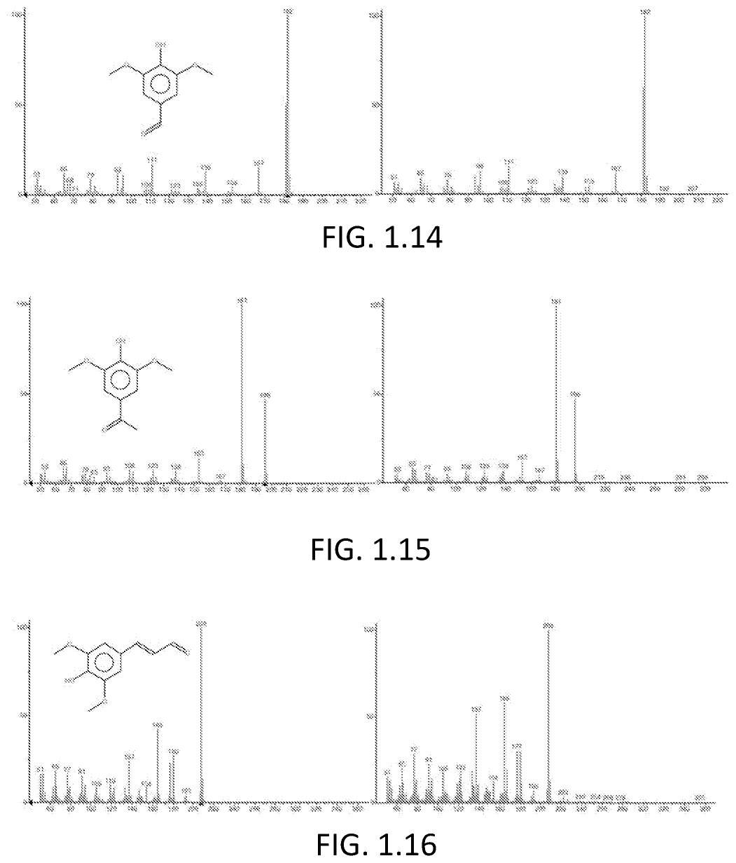

FIG. 1.11 illustrate a mass spectrum comparison used for qualification of Guaiacol.

FIG. 1.12 illustrate a mass spectrum comparison used for qualification of Vanillin.

FIG. 1.13 illustrate a mass spectrum comparison used for qualification of Vanillic acid, ethyl ester

FIG. 1.14 illustrate a mass spectrum comparison used for qualification of Syringaldehyde.

FIG. 1.15 illustrate a mass spectrum comparison used for qualification of Acetosyringone.

FIG. 1.16 illustrate a mass spectrum comparison used for qualification of Sinapaldehyde.

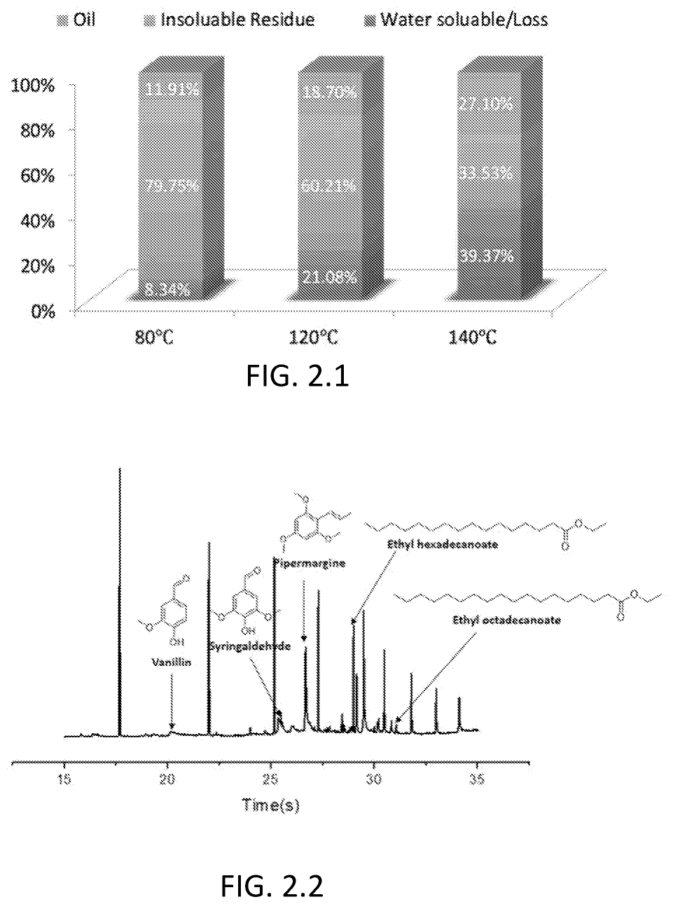

FIG. 2.1 illustrates the percentage distribution (weight basis) of the depolymerized products from Burcell MSW samples (solvent: 50/50 ethanol/water).

FIG. 2.2 illustrates GC-MS chromatogram of oil product by 140° C. in ethanol/water solution.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, materials science, mechanical engineering, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by volume, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequences where this is logically possible.

It must be noted that, as used in the specification and the appended claims, the singular forms “a,” “an,” and “the” include plural referents unless the context clearly dictates otherwise. Thus, for example, reference to “a compound” includes a plurality of compounds. In this specification and in the claims that follow, reference will be made to a number of terms that shall be defined to have the following meanings unless a contrary intention is apparent.

### DISCUSSION

In accordance with the purpose(s) of the present disclosure, as embodied and broadly described herein, embodiments of the present disclosure, in one aspect, relate to a method for depolymerizing lignin to produce other useful products. For example, low molecular weight aromatic and aliphatic hydrocarbons (e.g., hydrocarbons having 8 to 20 carbon atoms (C8 to C20 hydrocarbons)) as well as oil products can be produced using methods of the present disclosure. The method can include treatment of the lignin using a catalyst composition, where the catalyst composition comprises a persulfate salt and a transition metal catalyst. In one aspect, at least a portion of the lignin can be depolymerized to provide one or more of a C8 to C20 aromatic hydrocarbon, a C8 to C20 aliphatic hydrocarbon, or a combination thereof, where the pressure can be at 1 ATM and the temperature at less than about 120° C. In another aspect, at least a portion of the lignin can be depolymerized to provide one or more of a C8 to C20 aromatic hydrocarbon, a C8 to C20 aliphatic hydrocarbon, as well as oil products, where the pressure can be at 1 ATM and the temperature can be about 130° C. to 150° C.

It has been found that a low-cost biomimetic catalyst (Fe²⁺, H₂O₂) can effectively depolymerize lignin to low molecular weight aromatics and dicarboxylic acids with the conversion yield up to 78% in supercritical ethanol conditions (250° C., 7 MPa). However, in an effort to reduce the reaction harshness, a stronger oxidizer persulfate may be adopted instead of hydrogen peroxide to depolymerize lignin under milder conditions. Persulfate has stronger oxidation power than hydrogen peroxide, since the sulfate radical has higher oxidative power (E⁰=2.5-3.1 V) than hydroxyl radicals (E⁰=2.73 V).

Lignin comprises chains of aromatic and oxygenate constituents forming larger molecules that are not easily depolymerized. Herein, “lignin” and “lignin material” refer to a biomass material that is an amorphous three-dimensional energy-rich phenolic biopolymer. Lignin is typically deposited in nearly all vascular plants and provides rigidity and strength to their cell walls. The lignin polymeric structure is composed primarily of three phenylpropanoid building units (p-hydroxyphenylpropane, guaiacylpropane, and syringylpropane) interconnected by etheric and carbon-to-carbon linkages. Different types of lignin differ significantly in the ratio between these monomers. Non-limiting examples of lignin material can include plant lignin, woody lignin, lignin derived from agricultural and municipal waste, Kraft lignin, organosolv lignin, and combinations thereof.

The catalyst composition can include a persulfate. Generally speaking, persulfates have stronger oxidation power than hydrogen peroxide, as the sulfate radical has higher oxidative power (E⁰=2.5-3.1 V) than hydroxyl radicals (E⁰=2.73 V). In various aspects, the persulfate comprises S₂O₈²⁻. The persulfate can be a persulfate salt such as a Group 1A or 2A salt (e.g., Li, Na, Ca, K). The persulfate salt can be a sodium persulfate (Na₂S₂O₈), a potassium persulfate (K₂S₂O₈), or an ammonium persulfate ((NH₄)₂S₂O₈). Optionally, the persulfate is sodium persulfate.

The catalyst composition provides at least about 1.0 equivalents, or at least about 1.5 equivalents or at least about 2.0 or at least about 2.5 equivalents, or at least about 3.0 equivalents of persulfate. Molar equivalent to lignin content is used to calculate the persulfate loading. The amount of substance for lignin is calculated by an estimation on average molar mass of monomeric subunits.

The catalyst composition can include a transition metal catalyst. The transition metal catalyst comprises a compound comprising a one or more transition metals including, but not limited to, scandium (Sc), titanium (Ti), vanadium (V), chromium (Cr), manganese (Mn), iron (Fe), cobalt, (Co), nickel (Ni), copper (Cu), zinc (Zn), yttrium (Y), zirconium (Zr), Niobium (Nb), molybdenum (Mo), technicium (Tc), ruthenium (Ru), rhodium (Rh), palladium (Pd), silver (Ag), among others in groups 3 to 12 of the periodic table, and including alloys and salts thereof. In various aspects, the transition metal catalyst is a salt or metal oxide comprising one or more transition metals. In various aspects the transition metal is selected from a ferrous (Fe²⁺) compound or a cupric (Cu²⁺) compound. Optionally, the transition metal catalyst is selected from a ferrous sulfate salt or a copper sulfate salt. Optionally, the transition metal catalyst is ferrous sulfate heptohydrate (FeSO₄.7H₂O).

The catalyst composition can include about 0.1 mol % to about 10 mol % of the transition metal catalyst, on the basis of the persulfate loading in the catalyst composition. Optionally, the catalyst composition comprises about 1 mol % to about 9 mol %, or about 2 mol % to about 8 mol %, or about 3 mol % to about 7 mol %, or about 4 mol % to about 6 mol % of the transition metal catalyst, on the basis of the persulfate loading in the catalyst composition. Optionally, the catalyst composition comprises about 5% transition metal catalyst, on the basis of the persulfate loading.

The first composition can optionally include a phenol. It is theorized that the phenolic hydroxyl groups are a significant contributor to lignin recondensation. In various aspects, the addition of a phenol to the catalyst composition can significantly reduce lignin recondensation during the oxidation reaction, resulting in a reduction in the char formation present in the depolymerization product. In some aspects, the addition of phenol to the catalyst composition increases the yield of water-soluble chemicals present in the depolymerization product. The first composition comprises from about 5 wt % to about 15 wt % phenol, optionally from about 7 wt % to about 13 wt % phenol, optionally about 10 wt % phenol.

The depolymerization using the disclosed catalyst composition can be conducted under milder reaction conditions as compared to other oxidative depolymerization techniques. In various aspects the depolymerization step is performed at a pressure of about 1 atmosphere. In various aspects the depolymerization can be performed at a temperature of less than about 120° C., optionally from about 60° C. to about 100° C., optionally from about 70° to about 100° C., or optionally about 80° C. In some embodiments, the pressure can be increased and the temperature can be decreased to accomplish the desired depolymerization.

Alternatively, the can be performed at a temperature of greater than 125° C. to 160° C., about 130° C. to 150° C., about 135° C. to 155° C., or about 140° C. to produce a greater yield (e.g., about 20% or more, or about 30% or more) of oil products (e.g., C6-C20 aromatic hydrocarbons, such as those used for liquid fuel). In some embodiments, the pressure can be increased and the temperature can be decreased to accomplish the desired depolymerization and oil products.

The depolymerization can yield a depolymerization product comprising one or more low molecular weight aromatic hydrocarbon compounds. In some aspects, the depolymerization product comprises about 40 to 90 wt %, about 70 to 85 wt %, or up to about 76 wt % of the low molecular weight aromatics, on the basis of the total weight of the depolymerization reaction products. Optionally, the depolymerization product comprises greater than about 45 wt %, or greater than about 50%, or greater than about 55% or greater than about 60% or greater than about 65%, or greater than about 70%, by weight, of the low molecular weight aromatic or aliphatic hydrocarbons.

The depolymerization product can include less than about 20 wt % solid char, on the basis of the total weight of the reaction products. Optionally, the depolymerization product comprises less than about 19%, or less than about 18%, or less than about 17% or less than about 16%, or less than about 15%, or less than about 14%, or less than about 13%, or less than about 12%, or less than about 10%, or less than about 9%, or less than about 8%, or less than about 7%, or less than about 6% or less than about 5%, or less than about 4%, or less than about 3%, or less than about 2% solid char, on the on the basis of the total weight of the reaction products.

In various aspects, the depolymerization product comprises less than about 25% of water-soluble reaction products on the basis of the total weight of the reaction products. Optionally, the depolymerization product comprises less than about 21%, or less than about 20% or less than about 19%, or less than about 18%, or less than about 17% or less than about 16%, or less than about 15%, or less than about 14%, or less than about 13%, or less than about 12%, or less than about 10%, or less than about 9%, or less than about 8%, or less than about 7%, or less than about 6% or less than about 5%, or less than about 4%, or less than about 3%, or less than about 2% of water-soluble reaction products, on the basis of the total weight of the reaction products.

The depolymerization step can be completed with the described results within from about 2 hours to about 40 hours, optionally from about 12 hours to about 36 hours, optionally about 24 hours.

The products of the depolymerization can include aromatic monomers, oligomers (e.g., 2 monomers to 20 or more monomers, 2 monomers to 10 monomers, 2 monomers to 8 monomers, 2 monomers to 6 monomers, 2 monomers to 5 monomers, 2 monomers to 4 monomers, or 2 monomer to 4 monomers), or a combination thereof as well as C6 to C20 aliphatic monomers, oligomers, or combinations thereof. In some aspects, the products of the depolymerization include C6-C20 aromatic hydrocarbons, such as those used for liquid fuel such as jet fuel or marine fuel. In various aspects, the products of the depolymerization include high value aromatic monomers, aliphatic hydrocarbons. As described herein, the temperature can be adjusted up or down to produce the desired product(s) (e.g., increase the temperature to produce more oil products).

### EXAMPLES

Now having described the embodiments of the present disclosure, in general, example 1 describes some additional embodiments of the present disclosure. While embodiments of the present disclosure are described in connection with example 1 and the corresponding text and figures, there is no intent to limit embodiments of the present disclosure to these descriptions. On the contrary, the intent is to cover all alternatives, modifications, and equivalents included within the spirit and scope of embodiments of the present disclosure.

### Example 1

This Example aims to use a low-cost, biomimetic persulfate plus transition metal as the catalyst to effectively depolymerize lignin into monophenolic compounds under mild conditions (ambient pressure, temperature <100° C.). The Box-Behnken experimental design in combination with response surface methodology are applied to obtain optimized catalytical reaction conditions. The results show that this catalyst can depolymerize up to 99% of lignin dimers to mainly veratraldehyde. This reaction also successfully depolymerizes the industrial lignin to the phenolic oils (76%) with a high yield of monophenolic compounds. Quantum chemistry calculations at the density functional theory level indicate that the persulfate free radical attacks C_{β} to break the β-O-4 bond of lignin through a five-membered ring mechanism by passing less activation barrier than hydroxyl radicals. Gel permeation chromatography (GPC) and two-dimensional nuclear magnetic resonance spectroscopy (2D-NMR) demonstrates the effective cleavage of the β-O-4 bonds of lignin after depolymerization.

Lignin is the second most abundant polymer after cellulose in nature. It represents 35 wt. % of woody biomass^{[1]}. Lignin, as a byproduct from the pulp and paper industry and potentially polysaccharide-platform biorefineries, has been largely utilized as low-cost energy source through incineration in their power facilities despite its potential high-value as a source of aromatics. Commercial products derived from lignin only account for a small portion (2%) of total lignin production^{[2]}. For example, residue lignin from the pulp and paper industry has been converted either directly or through chemical modification into binders, heavy metal sequestrates, photo stabilizers or components of composites and copolymers and dispersants^{[3-6]}. Its application has been largely hindered by its heterogeneous molecular structure, complicated properties, composition deriving from different extraction processes and its high and scattered molecular weight^{[7]}. Native lignin is a crosslinked aromatic polymer comprised of p-hydroxyphenyl (H), guaiacyl (G), and syringyl (S) units with a molecular weight over 10,000^{[8]}. The molecular weight of industrial lignin is usually higher than that of native lignin due to the condensation reactions that occur during biorefinery or extraction processes. Therefore, it is necessary to depolymerize lignin to low-molecular weight aromatics as the promising precursors of liquid fuel (C6-C20)^{[9]} (e.g., jet fuel, marine fuel), aliphatic hydrocarbons, or high-value aromatic monomers. The value-added byproducts from lignin could also provide an extra asset in the polysaccharide-based biorefinery process.

Numerous routes^{[1,8-11]} have been investigated to depolymerize lignin or lignin model compounds into low-molecular-weight aromatics. These routes include thermal chemical cracking^{[12,13]}, catalytic reduction (hydrogenolysis)^{[1,15]}, hydrolysis^{[16]}, catalytic oxidation^{[17]}, photocatalysis^{[10]}, and enzymolysis^{[18]}. Lignin could be valorized by pyrolysis with or without the addition of catalysts, namely through hydrodeoxygenation and aqueous phase reforming^{[9,10,19-23]}. For example, Trenton et al. reported a complete destruction of lignin β-O-4 linkage using Pd/Zn/C as synergistic catalysts to depolymerize lignin model compounds at a pressure of 500 psi hydrogen and a reaction temperature of 225° C.^{[24]}. Li et al.^{[25]} improved the catalytic system by introducing Ru@N-doped carbon catalysts to achieve higher catalytic activity and better monomer yield. The introduction of protection groups^{[26,27,28]} is effective to stabilize lignin before hydrogenolysis and improve monomer yields. Additionally, lignin has been hydrolytically depolymerized by acid^{[29]}, alkaline^{[30-32]}, and supercritical water^{[31,33,34]} or ethanol^{[35,49]}. Deuss et al.^{[29]} reported that an extremely-strong trifluoromethanesulfonic acid could effectively convert lignin model compounds into monomers through the cleavage of C—O bonds^{[36]}. Saumya et al.^{[33]} conducted a base catalyzed lignin depolymerization method using dimethyl carbonate cesium carbonate as a catalyst at a high temperature to deconstruct the β-O-4 linkage of lignin. Most of these thermal chemical depolymerization methods are effective but perform under harsh reaction conditions (high temperature, high pressure) and require strong acids or complicated metal catalysts.

Oxidative depolymerization is a promising method to convert lignin to aromatic monomers, oligomers, or highly functional platform chemicals with potential applications in hydrocarbon fuels, bioplastic precursors, food, and pharmaceuticals^{[37]} in a relatively mild conditions. Various oxidizing agents such as organocatalyst (e.g. (2,2,6,6-tetramethylpiperidin-1-yl) oxyl, also known as TEMPO, organometallic catalysts, complicated biomimetic oxidizers (e.g. metalloporphyrins), and enzymatic catalysts^{[38]} have been extensively studied for lignin depolymerization. Organocatalyst TEMPO oxidized lignin has proven to be much easier for depolymerization than untreated lignin^{[39,40,41]}. Other oxidative depolymerization reactions focuses on the cleavage of C_{α}H—OH using the O₂/NaNO₂/DDQ/NHPI system^{[42]}, an electrocatalytic oxidation method^{[43]}, a Zn/NH₄Cl catalyst in methoxyethanol/H₂O solvent^{[23]}, and a complex vanadium catalyst in CD₃CN solvent^{[44]}. Complicated biomimetic catalysts including organocatalysts, organometallic catalysts and metalloporphyrin oxidizers face more challenges compared to enzymatic catalysts. They rapidly lose their reactivity and are difficult to recycle. Molecular oxygen and hydrogen peroxide are promising catalysts due to their environmentally-benign nature and cost-effectiveness. However, their oxidation capacity is relatively low and they need the aid of more complicated catalysts or more harsh reaction conditions. For example, Mitchell et al.^{[45]} adopted a photochemical method using 1,4-hydroquinone, a copper nanoparticle electron transfer mediator and oxygen to cleave the C—C linkage of the lignin model compound at room temperature and a reaction time up to 88 hrs, but the conversion yield was less than 30 wt. %. Mottweiler et al.^{[46]} used molecular oxygen with transition-metal-hydrotalcites (HTc) and V(acac)₃/Cu(NO₃)₂.3H₂O (acetylacetonate) mixtures to effectively depolymerize lignin model compounds to mainly veratric acid. Gregorio et al.^{[47]} coupled ionic liquid with a vanadium-based polyoxometalate to depolymerize lignin under oxygen-rich condition. The free radical related depolymerization reaction was extensively documented under normal physiological circumstances^{[48]}. Our previous work found that a low-cost biomimetic catalyst (Fe³⁺, H₂O₂) could effectively depolymerize lignin into low-molecular-weight aromatics and dicarboxylic acids with a conversion yield of up to 78%^{[49,52]} in supercritical ethanol condition (7 MPa, 250° C.).

In this Example, we investigate the use of a strong and low-cost oxidizer sodium persulfate, instead of hydrogen peroxide, in combination with a ferrous catalyst to reduce the reaction harshness for lignin depolymerization process. Previous research has shown that persulfate is capable of degrading phenolic contaminants such as 2,4,4′-trichlorobiphenyl at the aid of quinone^{[22]}. To the best of our knowledge, persulfate-based biomimetic catalysts have not yet been used for lignin depolymerization. Therefore, in this study, we investigate the feasibility of persulfate combined with a transition metal ferrous as a biomimetic catalyst to depolymerize lignin into monophenolic compounds under mild reaction conditions (less than 120° C., atmospheric pressure), using both lignin model compounds (β-O-4 dimer) and industrial lignin as feedstocks. The combined catalyst composition, dosage, and other reaction parameters are optimized on the conversion rate of depolymerize lignin into low-molecular-weight aromatics using Box-Behnken experimental design^{[63]} and the response surface methodology^{[64]}. We also elucidate the lignin depolymerization mechanism of this biomimetic catalyst by the density function theory (DFT) using the Gaussian program package^{[65]} as well as the experiment to compare lignin structure before and after depolymerization using a series of instruments.

### Results and Discussion

### Depolymerization of Lignin Model Compounds Using Biomimetic Persulfate Oxidizer

In this example, a low cost but strong oxidizer in substitution of a Fenton catalyst (peroxide with Fe³⁺) is applied to depolymerize lignin into low-molecular-weight aromatics under mild conditions. It is known that persulfate free radical at the aid of transition metals has stronger oxidative power (E₀=2.5-3.1 V) than a Fenton catalyst (peroxide, E₀=1.9-2.85 V)^{[50,51,53]}, this substitution is proposed to mitigate the harshness of the lignin oxidative depolymerization reaction. The lignin dimer (1R,2S)-1-(3,4-Dimethoxyphenyl)-2-(2-methoxyphenoxy)-1,3-propanediol was used as the starting material to evaluate the effectiveness of a persulfate-based biomimetic catalyst and its depolymerization mechanism. This lignin dimer model compound represents the major linkage (e.g. β-O-4 linkage) of lignin. The catalyst involves the persulfate with transition metals (i.e. Ferrous) or ions to generate free radicals as reactive oxidants. The lignin dimer and lignin oxidative depolymerization reactions occur under relatively mild conditions (60-100° C., 1 atm). The results show that with optimized conditions (refer to the subsequent experimental design data), the persulfate-based catalyst effectively converts up to 99% of lignin dimers into aromatic monomers.

After the oxidative reaction, the phenolic oil in the water was extracted by ethyl acetate and analyzed by both GC-MS. The GC-MS data indicates the formation of one major aromatic chemical (veratraldehyde) in the depolymerized products (FIG. 1). It was determined that the yield of veratraldehyde increased as the lignin dimers decreased at each subsequent reaction time. The lignin dimer was completely consumed and veratraldehyde was detected from the GC-MS spectra as the main product remaining after 1-h reaction time. But some char was observed in the depolymerized sample with a high loading of the oxidizer.

### Optimization of a Lignin Dimer Depolymerization Process

The conversion yields of the lignin dimer from all the depolymerization reactions based on the Box-Behnken experimental design under different reaction parameters are shown in Table 1. Three factors including persulfate loading, reaction time, and the reaction temperature are chosen to optimize this reaction. Each factor is conducted at a high level (+1), a center level (0) and a low level (−1). Thirteen experimental runs are performed in triplicate to obtain the average conversion rate of the lignin model compounds under certain combinations of reaction parameters. The response surface methodology is then adopted to optimize the depolymerization reaction conditions and elucidate the correlative effects of the persulfate loading, reaction time and reaction temperature on the conversion yield of the lignin model compounds. The response factors and the correlated factors are fitted using quadratic modelling as shown in Table S1.

A quadratic polynomial equation is established to describe the conversion yield of lignin model compound based on different levels of reaction factors (reaction parameters):

Y=89.2+10.8625x₁+16.8375x₂+27.075x₃−23.85x₃²

Y denotes the predicted conversion rate of the model compound. x₁, x₂, x₃ denote the coded level of persulfate loading, reaction time, and temperature, respectively.

As shown in the Table S1, Fisher's F-test with a low probability value demonstrates the polynomial model is highly significant. The F statistic is significantly greater than the corresponding tabulated F value (F_{0.05,9,5}=3.4817) at 5%, which indicates that the model explains the most possible variances of the reaction parameters. The coefficients with a P-value larger than 5% are insignificant and removed from the equation. Furthermore, we compare and plot the observed conversion rates of lignin model compound and predicted values, shown in FIG. 2. The data points of the predicted values are distributed evenly around the fitted line, indicating that the equation accurately describes the depolymerization reaction of lignin model compound. In addition, the predicted regression model has a high coefficient of determination (R=0.9485), which confirms the accuracy of the model.

TABLE 1
Box-Behnken design with three independent variables

| | Persulfate loading | | Time | | Temperature | | Conversion |
|---|---|---|---|---|---|---|---|
| | (Equivalent Ratio) | | (min) | | (° C.) | | (%) |
| | x₁ | X₁ | x₂ | X₂ | x₃ | X₃ | Y₀ |
| Entries | (coded) | (uncoded) | (coded) | (uncoded) | (coded) | (uncoded) | (Observed) |
| 1 | −1 | 1 | −1 | 30 | 0 | 80 | 29.8 |
| 2 | 1 | 2 | −1 | 30 | 0 | 80 | 68.6 |
| 3 | −1 | 1 | 1 | 120 | 0 | 80 | 76.9 |
| 4 | 1 | 2 | 1 | 120 | 0 | 80 | 98.9 |
| 5 | −1 | 1 | 0 | 60 | −1 | 60 | 17.6 |
| 6 | 1 | 2 | 0 | 60 | −1 | 60 | 22.1 |
| 7 | −1 | 1 | 0 | 60 | 1 | 100 | 77.0 |
| 8 | 1 | 2 | 0 | 60 | 1 | 100 | 98.6 |
| 9 | 0 | 1.5 | −1 | 30 | −1 | 60 | 28.5 |
| 10 | 0 | 1.5 | 1 | 120 | −1 | 60 | 43.6 |
| 11 | 0 | 1.5 | −1 | 30 | 1 | 100 | 55.3 |
| 12 | 0 | 1.5 | 1 | 120 | 1 | 100 | 97.5 |
| 13 | 0 | 1.5 | 0 | 60 | 0 | 80 | 89.2 |

FIG. 3 illustrates the contour and response surface plots of any two reaction parameters (persulfate loading, reaction time and reaction temperature) with the third parameter remaining at center level. It is observed that increasing the level of persulfate loading, reaction time, and reaction temperature increases the corresponding conversion yield of lignin dimer to monomers up to 99%. This result fits well with the aforementioned regression models. The surface response diagram can be used to optimize the reaction parameters to approach the maximum conversion yield of lignin dimer under harsh depolymerization reaction conditions. Surface response methodology has been applied to perform process optimization.

Table 2 shows the stationary point of the response surface. Herein we select the stationary point calculated by the model to help determine the optimal reaction conditions. The optimized conditions from the calculation is a 95° C. reaction temperature, 2.0 EQ persulfate loading with for 5% ferrous catalyst loading, and 129.6 min of reaction time.

TABLE 2
Stationary point of response surface for the
optimized reaction parameters

| | Un- | | Un- | | Un- |
|---|---|---|---|---|---|
| Persulfate | coded | Time | coded | Temperature | coded |
| Coded | (Eq) | Coded | (min) | Coded | (° C.) |
| 0.4101512 | 1.7 | 1.111162 | 129.6 | 0.762191 | 95.2 |

### Mechanism of Lignin Dimer Depolymerization

In nature, a reduced transition metal (manganese) catalyzes H₂O₂ to form reactive hydroxyl radicals to hydroxylate and therefore depolymerize lignin via demethoxylation. This study uses biomimicry, wherein, similar to hydrogen peroxide, a transition metal catalyzes persulfate to form persulfate free radicals with stronger oxidative power than hydrogen peroxide free radicals. The persulfate free radicals attack the lignin dimer to cleave either α-β and β-O-4 bonds to produce monomeric veratraldehyde under mild conditions (1 atm, 80° C.), in comparison with high pressure and high temperature conditions (7 MPa, 250° C.) using Fenton catalyst. In order to further understand the differences between these two free radicals' efficacy in the cleavage of bonds within the lignin model compound 1-(3,4-dimethoxyphenyl)-2-(2-methoxyphenoxy)-1,3-propanediol. —OH radical was first applied to attack different groups and targeted at the optimized medium structures, the transition states, and the products of the radical reactions. FIG. 4 summarizes the activation barriers, table S2-S16 demonstrate the energies and structural coordinates for all species.

FIG. 4 shows that both the C_{α}-C_{β} bond and β-O-4 bond are comparatively difficult to break. The activation barrier for C_{α}-C_{β} cleavage is 40 kcal/mol when .OH attacks from the C_{α}-side, and is 43 kcal/mol when .OH attacks from the C_{β}-side, whereas the activation barrier for the β-O-4 bond cleavage is 41 kcal/mol. Therefore, it is reasonable that this study to employ a stronger oxidant persulfate in order to obtain mild reaction environment. Based on the aforementioned experimental results, herein, we propose a new free radical reaction mechanism. In the presence of Fe²⁺, one persulfate ion is anion (SO₄⁻.).

The sulfate radical anion (SO₄⁻.) can certainly react as .OH but with higher oxidative powder. Previous research shows that NaIO₄ can break 1,2-diol structure and produce aldehydes^{[54]}, and the C_{α}-C_{β} unit is close to 1,2-diol (one OH group on C_{α} and one OR group. Therefore, a similar five-membered ring reaction mechanism is proposed in FIG. 5. First, the α-OH group attacks the sulfur atom of SO₄⁻., which also induces the H⁺ transfer of the OH group to O of SO₄⁻. at the same time. Then the SO₄⁻. radical anion attacks C_{β} to break the β-O-4 bond, so that the five-membered ring intermediate is also created. Finally, the five-membered ring facilitates the breaking of the C_{α}-C_{β} bond to produce the main product veratraldehyde (3) as shown in our GC-MS results. This result also meets the oxidative theory for using NaIO₄ to break down the C—C bond of 1,2-diols. Regarding the lignin model compound (R₁═CH₃ and R₂═H), the key steps in the proposed mechanism with M062X/6-311G** level of theory was also calculated. Indeed, the β-O-4 bond cleavage only needs to pass an activation barrier of 25.8 kcal/mol, which is lower than the barrier .OH radicals needed to break the same bond (41 kcal/mol). In addition, the cleavage of the C_{α}-C_{β} bond only needs to pass a barrier of 12.1 kcal/mol, which is much lower than the reaction barriers with .OH radicals (40-43 kcal/mol). This results further approves the stronger oxidation power of persulfate.

The radical 4 is responsible for the formation of solid char from further condensation reactions. During our experiment, it was observed that, when a large amount of persulfate (over 5 equivalent) was added and a higher temperature (120° C.) was applied, dark brown solid residues were observed during the reaction and their size increased with longer time or enhanced temperature. It is obvious that the incomplete or over-oxidative reactions leads to the formation of char due to the formation of radical 4. As shown in FIG. 6, the added phenol could reduce the char formation. It can be explained that the phenol may block radical 4 from this side reaction and reduce the char formation. However, the added phenol does not affect the yield of veratraldehyde 3. This result agrees well with our proposed mechanism. In the proposed mechanism (FIG. 5), the veratraldehyde is not directly derived from the radical 4, which is responsible for the formation of char. The formation of radical 4 directly comes from the cleavage of the β-O-4 bond, which also agrees well with the further NMR analysis. Since sulfur has empty 3d-orbitals, the hypervalent configuration (beyond the octet rule) for S atom in 2 and 2′ is allowed. However, we also find that the five-membered ring would experience more strain than the corresponding IO4. species because of the smaller size of the S atom compared to the I atom, and we discover one long S—O bond (1.877 Å) in 2′.

### Depolymerization of Industrial Lignin

A fundamental understanding of the reaction mechanism, catalyst efficiency and the optimized reaction conditions are based on the lignin model compound, which provides the theoretical and practical support for the depolymerization of industrial lignin extracted from the biorefinery or pulping process. The aforementioned lignin dimer represents the major linkage (e.g. β-O-4 linkage) of lignin, and therefore it simulates the cleavage of the major interlinkage between the aromatic rings and the effectiveness of the catalyst under the appropriate reaction conditions. Lignin reclaimed from a biorefinery process using extraction composed more complicated molecular structure and a set of properties depending on the biomass species, pretreatment conditions of cellulosic biomass, and the isolation process. Industrial lignin usually requires much harsh conditions to break down the inter-linkages, and the catalyst behaves differently with the lignin dimer and native lignin. In this study, two types of industrial lignin including organosolv lignin and alkali lignin were used as feedstocks to evaluate the efficacy of persulfate as the catalyst. To determine the optimized reaction conditions for industrial lignin, we firstly conducted the depolymerization reactions of lignin dimers near to the optimized reactions condition from the surface response methodology. It was found that the optimized reaction conditions for lignin depolymerization using lignin dimer as the feedstock considering the economic cost of the process are 2 h reaction time, 80° C. reaction temperature, 2.0EQ persulfate loading, and 5% Ferrous catalyst loading (based on persulfate), which could achieve the depolymerization of 99% of the lignin dimer. This reaction condition was used for the depolymerization reaction of the industrial lignin. Then, we investigated that the reaction time should be prolonged to 24 hrs. to depolymerize the industrial lignin due to its complicated interlink bonds and structure. The ethanol/water solution (50/50 wt. ratio) was applied to improve the solubility of the two types of industrial lignin.

Two types of industrial lignin (organosolv. and alkali lignin) was depolymerized in an optimized reaction condition determined by both the surface response calculation and the actual experimental result in considering the lowest economic cost. FIG. 6 displays the yields of the depolymerized products from two types of industrial lignin. The depolymerization of organosolv lignin yielded 76.2% oil product, 15.4% solid char, and 8.4% water solubles. The depolymerization of alkali lignin at the same conditions yielded 46.7% oil product, 31.2% solid char, and 22.1% water solubles. The GPC data (FIG. 7,8) demonstrate that the average molecular weight decreases from 2200 Da to 1300 Da for organosolv lignin and from 1980 Da to 1560 Da for alkali lignin, respectively. Thus, organosolv lignin is less recalcitrant than alkali lignin during the persulfate-based lignin depolymerization process although organosolv lignin has a higher molecular weight than alkali lignin. Since the organosolv lignin was treated in ethanol at a moderate pretreatment condition, it was condensed less. The alkali pretreatment process usually produces some hard-to-degrade lignin oligomers that prevent further degradation. In addition, alkali lignin produced more solid char, which further explains its higher recalcitrance. The water solubles are side products of the lignin depolymerization process and typically made of organic acids such as dicarboxylic acids and acetic acids. Additionally, phenol was added into the organosolv lignin to further improve the depolymerization reaction. Phenol has been reported as an effective stabilizer to block the phenolic OH group in lignin during the depolymerization process^{[55-57]} because the phenolic hydroxyl group is the main cause for lignin re-condensation, especially when lignin is demethoxylated by free radicals. The addition of 10% phenol significantly reduced the char formation from organosolv lignin from 15.4% to 5.7% and increased the yield of water-soluble chemicals, but didn't change the total oil yield. This result indicates that phenol could effectively prevent lignin re-condensation during the oxidation depolymerization reaction. The mechanism for the formation of water-solubles requires further elucidation.

FIG. 9 shows the GC-MS spectrum of depolymerized lignin oil products from two types of industrial lignin. Herein guaiacol was used as an internal standard to calibrate all the monomer compounds. All the mass spectrum of pure chemicals for the GC-MS measurement are shown in Figure S7. Monomers derived from p-hydroxyphenyl, guaiacyl, and sinapyl units were detected from the oil product of organosolv lignin. It was observed that the yield of products derived from syringyl residues was higher than the yield of products derived from guaiacyl and p-hydroxyphenyl residues, which was due to a high ratio of S unit in the organosolv lignin. Also, the aldehyde structure of the monomer products further agreed with the aforementioned free radical reaction mechanism using lignin model compounds (Table 3). The total yield of monophenolic compounds in organosolv. lignin is 26.74% including guaiacol, vanillin, vanillic acid ester, syringaldehyde, 3,4,5-thimethoxy-benzaldehyde, acetosyringone and sinapaldehyde. The oligomers after 30-min retentions are also observed. The GC-MS spectrum also show that the alkali lignin forms less monophenolic compounds, which agreed well with the GPC data.

TABLE 3
Yields of monomers from depolymerization
of organosolv lignin

| Name | Yields (w/w oil product) |
|---|---|
| Guaiacol | 6.38% |
| Vanillin | 4.49% |
| Vanillic acid, ethyl ester | 1.23% |
| Syringaldehyde | 14.15% |
| Benzaldehyde, | 0.92% |
| 3,4,5-trimethoxy- | |
| Acetosyringone | 3.27% |
| Sinapaldehyde | 2.67% |
| Total | 26.74% |

### HSQC 2D-NMR Analysis Before and After Lignin Depolymerization

In order to further understand how the persulfate catalyst breaks down the industrial lignin interlinkages, the HSQC 2D-NMR was used to characterize the lignin structure before and after depolymerization for both organosolv and alkali lignin. FIG. 10 shows the 2D-NMR spectra corresponding to the aromatic region (δ_{C}/δ_{H} 90-160/6.0-8.0) and aliphatic region (δ_{C}/δ_{H} 50-90/2.0-6.0) for two different phenolic oils. There is no significant difference between the reacted sample and the standard sample in the aromatic area δ_{C}/δ_{H} 90-160/6.0-8.0, which supports the conclusion that the reaction doesn't deconstruct the aromatic structure of lignin monomers. The side chain areas are compared to analyze the reaction on the lignin linkages.

As shown in FIG. 10 and Table 4, the crosspeaks corresponding to the α ρ and the γ position of β-O-4 or other lignin side chain structures are distributed in the aliphatic region, or the δ_{C}/δ_{H} 50-90/2.0-6.0 region. The signal of δ_{C}/δ_{H} 71/4.7 is ascribed to the α-OH of a C3 β-O-4 structure. The signal covering the region of δ_{C}/δ_{H} 80-88/4.4-3.9 is ascribed to the β carbon hydrogen bonds of the β-O-4 linkage. With different lignin units (G/H or S), the signal shifts within the area and generated a wide signal region. The signal of the γ position within the β-O-4 linkage is found at the δ_{C}/δ_{H} 58-62/3.0-3.8 region. The signal of the depolymerized structure, which is the γ position signal of the monomer, is found at the δ_{C}/δ_{H} 58-62/3.9-4.2 region. The most prominent peak appears at δ_{C}/δ_{H} 54-57/3.0-4.0 and it is assigned to the methoxyl group. The signal of β-β linkage is also observed in the spectrum. δ_{C}/δ_{H} 85/4.6 is ascribed to the α position of the β-β structure. δ_{C}/b_{H} 55/3.0 corresponds to the β position. The γ position of a β-β linkage is found at the region δ_{C}/δ_{H} 70-73/4.0.

Comparing the spectra of the lignin before and after the reaction reveals that signal intensity corresponding to the β-O-4 linkage is significantly reduced after the depolymerization reaction, which agrees well with the cleavage of the β-O-4 linkage by sulfate free radicals in our lignin model study. HSQC spectroscopy also provides detailed information about the depolymerized products. The signal that belongs to the γ position signal of the monomer increases after the reaction, indicating the cleavage of the lignin monomer side chains. The intact signal representative of the β-β linkage in the HSQC spectrum after the reaction provides the evidence for the selective cleavage of the β-O-4 linkage by the free radicals instead of the β-β linkage.

TABLE 4
Heteronuclear single quantum coherence (HSQC)
2D nuclear magnetic resonance (NMR) spectra peak list

| Label | δ_{C}/δ_{H} | Assignment |
|---|---|---|
| Bβ | 55/3.0 | C_{β}-H in β-β linkages (B) |
| Methoxyl | 54-57/3.0-4.0 | C-H in methoxyl group |
| Cγ | 58-62/3.9-4.2 | C_{γ}-H in monomer side chain (C) |
| Aγ-OH | 58-62/3.0-3.8 | C_{γ}-H in β-O-4 linkages (A) |
| Bγ | 70-73/4.0 | C_{γ}-H in β-β linkages (B) |
| Aα-OH | 71/4.7 | C_{α}-H in β-O-4 linkages (A) |
| Aβ(G/H) | 80-83/4.4-4.2 | C_{β}-H in guaiacyl or |
| | | p-hydroxyphenyl β-O-4 |
| | | linkages (A) |
| Aβ(S) | 85-88/4.2-3.9 | C_{β}-H in syringyl β-O-4 |
| | | linkages (A) |

### Conclusions

The objective of this study is to apply a biomimetic persulfate-based catalyst to effectively depolymerize both a lignin model compound and industrial lignin into monomeric aromatic chemicals under mild reaction conditions. The results show that a persulfate-based catalyst significantly decreases the need for harsh reaction conditions due to its higher oxidation power compared with a hydrogen peroxide-based biomimetic catalyst. Persulfate in combination with transition metals effectively depolymerizes up to 99% of lignin dimers into monomers under mild reaction conditions (95° C., 1 atm). The catalyst also effectively degrades industrial lignin into monophenonic compounds with a total yield of 26.74% under mild reaction conditions (80° C., 1 atm). The Gibbs free energy calculation by DFT shows that both the cleavage of β-O-4 and C_{α}-C_{β} bonds pass a lower activation energy barrier for persulfate free radicals than hydrogen peroxide. The HSQC 2D-NMR spectra confirm the deconstruction of the β-O-4 linkage in the industrial lignin. However, a small amount of unstable monomers are produced via the free radical reaction which induce solid char formation, thereby warranting careful attention to avoid overdosing the persulfate catalyst.

### Experimental Section

### Materials

In this study, sodium persulfate (>99.0%) and hardwood alkali lignin were purchased from Sigma-Aldrich Inc. (St. Louis, Mo., USA). Ferrous sulfate heptahydrate (>99.0%) was purchased from Thermo Fisher Scientific Inc. (Waltham, Mass., USA). Hardwood organosolv lignin was kindly provided by Professor Pan from the University of Wisconsin-Madison. The lignin model compound (1R,2S)-1-(3,4-Dimethoxyphenyl)-2-(2-methoxyphenoxy)-1,3-propanediol (CAS number 7572-98-7) was synthesized using the modified procedure according to Buendia et al.^{[58]} (see the SI for its synthesis)

### General Procedures for the Catalytic Oxidation of Lignin Model Compound (β-O-4 Dimers)

The oxidative degradation of lignin model compounds (β-O-4) with sodium persulfate under mild conditions was investigated. In a typical reaction, 10 mL of aqueous solution of sodium persulfate was added into the glass vial. The lignin model compound (10 mg) with or without the ferrous aqueous solution (5% based on the molar ratio to sodium persulfate) were then added into the reaction vial. The reaction vial was sealed and placed in an oil bath and stirred at 500 rpm at the setting reaction temperature (60-100° C.) for 30-120 mins. After the reaction was terminated, the product underwent a liquid/liquid extraction using organic solvent ethyl acetate for further analysis. The conversion yield was calculated based on the percentage of the reactant reduction during each reaction for lignin model compound.

### General Procedures for the Catalytic Oxidation of Industrial Lignin

Two types of lignin including alkali lignin and organosolv lignin were used as the starting materials for the free radical oxidation reaction. In a typical run, a 100 mg lignin sample was dissolved in 10 mL of ethanol/water (1:1 w/w) solution and then loaded into a sealed glass tube reactor. Persulfate (2.0 equivalent based on lignin weight) and a 5% molar fraction of ferrous catalyst were then added to the reactor and the temperature was increased to 80° C. to initiate the reaction. The reaction was maintained for 24 hrs because lignin has a much higher molecular weight and a more complicated molecular structure than its model compound. During the reaction, samples were collected from the sealed tube after 3 h and 6 h of reaction for gel permeation chromatography (GPC) analysis. Upon completion, the product then underwent liquid/liquid extraction three times using ethyl acetate (EtOAc) and was then washed with water and saturated sodium chloride solution, following the same procedure as was used with the lignin model compounds. The organic liquid was evaporated in a rotary evaporator to remove the solvent to produce the organic oil. After the removal of the organic liquid, the remainders in the reactor were completely washed with water. All the liquid was collected and then filtered through a weighed filter paper to collect insoluble char. The control experiment was performed using the same protocol but in the absence of persulfate. When phenol was used as the blocking agent, 50 mg phenol was added to the system to evaluate how it affected the lignin oxidative depolymerization process. The yields of phenolic oil and solid char were calculated based on the mass of raw material lignin.

### Gas Chromatography-Mass Spectrometry (GC-MS) Measurement

Gas chromatography-mass spectrometry (GC-MS, GC-Agilent 7820 and MS-Agilent 5977) was used to determine the chemical composition of the phenolic oil product. Helium was used as the carrier gas at a flow rate of 1 mL/min with a 1:20 split ratio. For each sample, 10 mg of oil product was dissolved in 1 mL of organic solvents, dried with magnesium sulfate anhydrous and filtered through a 0.22 μm syringe filter. An Agilent HP-5MS 5% Phenyl Methyl Silox 30 m×250 μm×0.25 μm column was set in the GC oven to separate the sample and the oven temperature was ramped up from 50° C. to 280° C. The energy of the electrons was set at 70 eV to generate MS spectra comparable with the online database.

The GC-MS result was calibrated with either an internal standard method or an external standard curve. In a typical measurement, 0.1 mg/mL (100 ppm) of guaiacol was added to the vial with the sample as an internal standard to determine the concentration of each component in the oil product. For a model compound test, the standard curve for the reactant (β-O-4 dimer and monomer model compound) and all possible depolymerized products (e.g. veratrylaldehyde, guaiacol) were generated prior to the test for qualification purpose. The conversion yield was calculated based on the percentage of the reactant reduced during each lignin model compound reaction using the concentration obtained from GC-MS.

### Heteronuclear Single Quantum Coherence Spectroscopy (HSQC) Measurement

The HSQC ¹³C-¹H Correlation NMR measurement was performed using deuterated dimethyl sulfoxide (DMSO-d₆) as the solvent. For each sample, 500 μL of 99.9% DMSO-d₆ was used to dissolve 35 mg of the phenolic oil product from the lignin depolymerization process. The heteronuclear single quantum coherence (HSQC) NMR spectra were recorded on a Bruker Avance II 600 MHz equipped with a 5 mm TXI cryoprobe at 27° C. MestReNova software was used to interpret the 2D-NMR spectrum. Both the side chain region and aromatic areas were recorded and crosspeaks were assigned with literature records. All the reactions were triplicated, and the standard error was calculated for each measurement.

### Gel Permeation Chromatography (GPC) Measurement

The gel permeation chromatography (GPC) was used to analyze the molar mass distribution of the lignin sample and depolymerized oil products. Three individual pore-size organic GPC columns (Agilent PLgel Sum 10,000 Å, Agilent PLgel 5 μm 100 Å and Agilent PLgel 10 μm Mixed-B) were connected in a series and carried by an Agilent 1260 HPLC system. A 5 mg lignin sample or phenolic oil product was dissolved in 1 mL HPLC grade tetrahydrofuran (THF), dried with magnesium sulfate anhydrous and filtered through a 0.22 μm syringe filter. The sample was then injected into the column for analysis.

### Computational Methods

The relative Gibbs Energy (i.e., energy diagram) for the proposed lignin dimer depolymerization mechanism and all the calculations including transition state search were performed using density functional theory (DFT) with the Gaussian program package. The MMFF94 force field and the Global-MMX steric energy minimization program were utilized in order to determine possible conformers of the lignin model compounds. The compounds were optimized for stability at theoretical model level M06-2X/6-311G** with SMD water. M06-2X is density functional according to Zhao and Trular^{[60]} and is shown to have excellent agreement with known benchmark tests^{[61]}, and the SMD is a universal solvation model which calculates free energy changes in solutions based on solute electron density and a continuum model of the solvent^{[62]}. The transition states for the chemical reactions between reactants and products were also optimized at the M06-2X/6-311G** level with SMD water. The frequencies were calculated at the same level to obtain enthalpy and free energy and confirm whether the structures were the minima (without imaginary frequencies) or the transition states (with only one imaginary frequency).

### Box-Behnken Experimental Design and Response Surface Methodology for Lignin Depolymerization Reaction

Response surface methodology (RSM) was used to explore relationships between the conversion yield and three explanatory variables (reaction time, reaction temperature and persulfate loading). The Box-Behnken design^{[64]} was adapted to decide the number of experimental points and to construct the response surface of this set of the experiment. Each variable was considered as an individual factor and was placed at three equally spaced levels, coded as −1, 0, +1 for each level. With these assumptions, 13 entries were determined to be sufficient to fit a quadratic model.

### Supplemental Information

### Synthesis of Lignin Model Compound (Lignin β-O-4 Dimer)

Scheme S1 describes the synthesis of the lignin β-O-4 dimer model compound. In the first step, guaiacol (50 mmol), potassium carbonate (55 mmol) and ethyl bromoacetate (55 mmol) were added into a 250 mL flask. Acetonitrile (50 mL) was then added into the flask. The reaction was conducted at 60° C. for 24 hrs, accompanied by stirring. Water was added to quench the reaction at the end. The final product was extracted and purified with a silica gel column. In the second step, a round bottom flask was thoroughly cleaned and purged with nitrogen in a −78° C. acetone/dry ice cooling bath. Tetrahydrofuran (100 mL) was added and stirred. The product of the previous step, ethyl (2-methoxyphenoxy) acetate (30 mmol), lithium diisopropylamide (LDA) (30 mmol) and veratrylaldehyde (35 mmol) were then slowly injected into the reactor. The reaction was kept overnight at −78° C. After the reaction was finished, several drops of 1% hydrochloric acid was added to quench the reaction. The product was purified and reduced by sodium borohydride to give the final product (1R,2S)-1-(3,4-Dimethoxyphenyl)-2-(2-methoxyphenoxy)-1,3-propanediol. Comparison of mass spectrum between standards and products are shown in FIGS. 1.11-1.16.

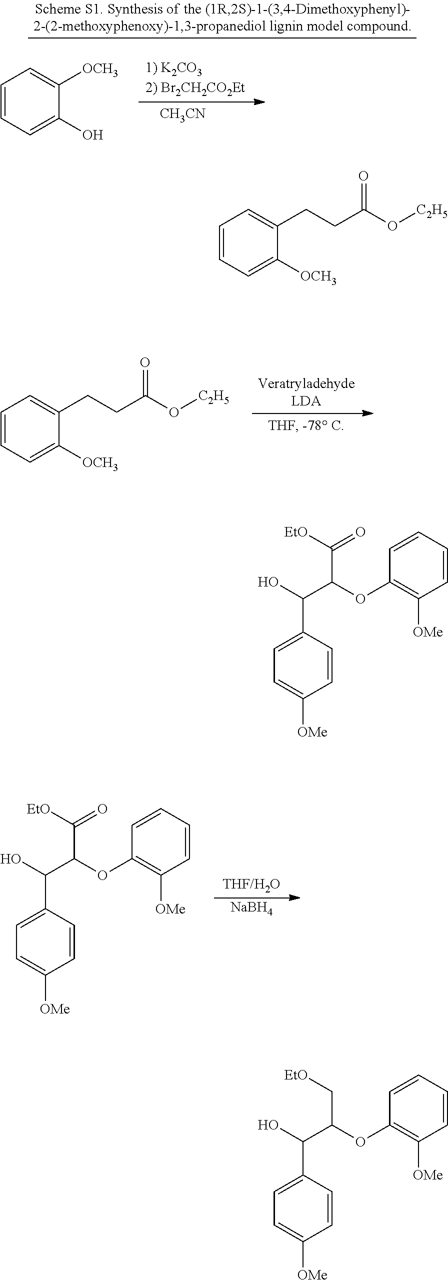

### Response Surface Model

TABLE S1
Statistics of predicted response surface model

| | | Statistics | Stan- | | |
|---|---|---|---|---|---|
| Factor | | Coef- | dard | | |
| (coded) | | ficient | Error | t value | P value |
| Intercept | | 89.2 | 6.2284 | 14.3215 | 2.992E−05 |
| x₁ | | 10.8625 | 3.8141 | 2.848 | 0.0359083 |
| x₂ | | 16.8375 | 3.8141 | 4.4145 | 0.0069271 |
| x₃ | | 27.075 | 3.8141 | 7.0987 | 0.0008595 |
| x₁x₂ | | −4.2 | 5.3939 | −0.7787 | 0.4714096 |
| x₁x₃ | | 4.275 | 5.3939 | 0.7926 | 0.4639645 |
| x₂x₃ | | 6.775 | 5.3939 | 1.256 | 0.2645956 |
| x₁² | | −11.525 | 5.6142 | −2.0528 | 0.0952986 |
| x₂² | | −9.125 | 5.6142 | −1.6253 | 0.1650174 |
| x₃² | | −23.85 | 5.6142 | −4.2482 | 0.0081061 |
| Multiple | R- | 95.38% | | | |
| squared: | | | | | |
| Adjusted | R- | 87.07% | | | |
| squared: | | | | | |
| F-statistic: | | 11.47 | | | |
| p-value: | | 0.007599 | | | |

### Lists of Structural Coordinates

TABLE S2
Energies at M062X/6-311G** level with SMD water (a.u.)

| | E | ZPE | H | G |
|---|---|---|---|---|
| 1-(3,4- | −1150.31 | 0.38 | −1149.90 | −1149.98 |
| dimethoxyphenyl)-2- | | | | |
| (2-methoxyphenoxy)- | | | | |
| 1,3-Propanediol | | | | |
| SO₄·⁻ | −699.10 | 0.01 | −699.08 | −699.12 |
| SO₃H⁻ | −624.55 | 0.02 | −624.53 | −624.56 |
| veratraldehyde | −574.54 | 0.18 | −574.35 | −574.40 |
| 2-(2- | −575.75 | 0.20 | −575.53 | −575.58 |
| methoxyphenoxy) | | | | |
| ethanol | | | | |
| part2-radical2 (α,β- | −535.82 | 0.16 | −535.65 | −535.70 |
| C-C cleavage-CH₃) | | | | |
| SO₃-CH₃⁻ | −739.08 | 0.06 | −739.01 | −739.05 |
| O₂ | −150.25 | 0.00 | −150.24 | −150.26 |
| 2,4-dimethoxy- | −649.80 | 0.18 | −649.60 | −649.65 |
| bezoic acid | | | | |
| SO₃-CH₂-CH₂-OH⁻ | −853.60 | 0.09 | −853.50 | −853.55 |
| Quinone | −381.40 | 0.09 | −381.30 | −381.34 |
| 2-OH-acetylaldehyde | −229.02 | 0.06 | −228.95 | −228.98 |
| 1 | −1849.32 | 0.40 | −1848.89 | −1848.98 |
| TS1-2 | −1849.27 | 0.40 | −1848.84 | −1848.93 |
| 2 | −1849.38 | 0.40 | −1848.95 | −1849.05 |
| 2′ | −1428.09 | 0.27 | −1427.80 | −1427.87 |
| TS2-3 | −1428.04 | 0.27 | −1427.76 | −1427.83 |
| 3 | −1428.15 | 0.26 | −1427.86 | −1427.93 |
| 4 | −421.29 | 0.13 | −421.16 | −421.20 |

TABLE S3
1-(3,4-dimethoxyphenyl)-2-(2-methoxyphenoxy)-1,3-
Propanediol coordinates (Ångström)

| Atoms | X | Y | Z |
|---|---|---|---|
| C | −2.8970 | −0.6668 | −0.2034 |
| C | −2.0132 | −0.1055 | −1.1406 |
| C | −0.7657 | −0.6600 | −1.3476 |
| C | −0.3565 | −1.7785 | −0.6186 |
| C | −1.2372 | −2.3463 | 0.2927 |
| C | −2.5057 | −1.8028 | 0.5000 |
| H | −0.0992 | −0.1865 | −2.0619 |
| H | −0.9329 | −3.2157 | 0.8637 |
| H | −3.1716 | −2.2626 | 1.2182 |
| O | −2.3992 | 1.0216 | −1.8356 |
| O | −4.0945 | −0.0378 | −0.0675 |
| C | −2.1189 | 2.2182 | −1.0972 |
| H | −1.0430 | 2.3075 | −0.9178 |
| H | −2.4607 | 3.0524 | −1.7081 |
| H | −2.6554 | 2.2161 | −0.1444 |
| C | 1.0724 | −2.2523 | −0.7270 |
| H | 1.4419 | −2.0744 | −1.7441 |
| C | 1.9899 | −1.4710 | 0.2287 |
| H | 1.6485 | −1.5928 | 1.2603 |
| C | 3.4451 | −1.9293 | 0.1425 |
| H | 3.7541 | −1.9621 | −0.9098 |
| H | 4.0627 | −1.1938 | 0.6596 |
| O | 1.9834 | −0.1029 | −0.1736 |
| C | 1.3085 | 0.8482 | 0.5431 |
| C | 1.6291 | 2.1773 | 0.1942 |
| C | 0.3468 | 0.6063 | 1.5125 |
| C | 0.9912 | 3.2329 | 0.8328 |
| C | −0.2946 | 1.6745 | 2.1460 |
| H | 0.0710 | −0.4073 | 1.7711 |
| C | 0.0256 | 2.9786 | 1.8100 |
| H | 1.2356 | 4.2546 | 0.5741 |
| H | −1.0474 | 1.4686 | 2.8974 |
| H | −0.4688 | 3.8099 | 2.2977 |
| O | 2.5697 | 2.3147 | −0.7826 |
| C | 2.9078 | 3.6410 | −1.1721 |
| H | 3.3308 | 4.1995 | −0.3329 |
| H | 3.6544 | 3.5402 | −1.9566 |
| H | 2.0333 | 4.1678 | −1.5630 |
| O | 1.2229 | −3.6230 | −0.3808 |
| H | 0.7373 | −4.1545 | −1.0224 |
| O | 3.6525 | −3.1773 | 0.7822 |
| H | 3.0170 | −3.7899 | 0.3886 |
| C | −5.0027 | −0.5738 | 0.8896 |
| H | −5.8872 | 0.0574 | 0.8482 |
| H | −5.2744 | −1.6017 | 0.6371 |
| H | −4.5739 | −0.5391 | 1.8945 |

TABLE S4
Free radical SO₄·⁻ coordinates (Ångström)

| Atoms | X | Y | Z |
|---|---|---|---|
| S | −0.0001 | 0.0841 | 0.0000 |
| O | −0.0001 | −0.9628 | −1.1020 |
| O | −0.0001 | −0.9628 | 1.1020 |
| O | 1.2222 | 0.8786 | 0.0000 |
| O | −1.2219 | 0.8788 | 0.0000 |

TABLE S5
Ion SO₃H⁻ coordinates (Ångström)

| Atoms | X | Y | Z |
|---|---|---|---|
| S | −0.1619 | −0.0004 | −0.3965 |
| O | 1.4783 | 0.0031 | 0.0577 |
| H | 1.5045 | 0.0005 | 1.0288 |
| O | −0.6687 | −1.2280 | 0.3032 |
| O | −0.6739 | 1.2256 | 0.3034 |

TABLE S6
Veratraldehyde coordinates (Ångström)

| Atoms | X | Y | Z |
|---|---|---|---|
| C | −0.9355 | −0.5372 | −0.1094 |
| C | −0.5020 | 0.7903 | −0.2972 |
| C | 0.8427 | 1.0917 | −0.2549 |
| C | 1.7840 | 0.0787 | −0.0407 |
| C | 1.3562 | −1.2353 | 0.1406 |
| C | 0.0053 | −1.5482 | 0.1087 |
| H | 1.1566 | 2.1206 | −0.4004 |
| H | 2.0900 | −2.0161 | 0.3029 |
| H | −0.3120 | −2.5725 | 0.2474 |
| O | −1.4326 | 1.7734 | −0.5434 |
| O | −2.2687 | −0.7289 | −0.1662 |
| C | −2.0493 | 2.2678 | 0.6526 |
| H | −1.2998 | 2.7319 | 1.2989 |
| H | −2.7788 | 3.0130 | 0.3408 |
| H | −2.5540 | 1.4612 | 1.1891 |
| C | 3.2122 | 0.4202 | −0.0128 |
| H | 3.4444 | 1.4880 | −0.1678 |
| O | 4.1046 | −0.3856 | 0.1642 |
| C | −2.7589 | −2.0550 | 0.0322 |
| H | −3.8409 | −1.9828 | −0.0432 |
| H | −2.3784 | −2.7272 | −0.7400 |
| H | −2.4806 | −2.4250 | 1.0215 |

TABLE S7
2-(2-methoxyphenoxy) ethanol coordinates (Ångström)

| Atoms | X | Y | Z |
|---|---|---|---|
| C | 2.1760 | 0.3567 | −0.0203 |
| H | 2.2404 | 0.9867 | −0.9131 |
| C | 3.2939 | −0.6674 | −0.0189 |
| H | 3.2022 | −1.3146 | 0.8588 |
| H | 3.2273 | −1.2874 | −0.9139 |
| O | 0.9525 | −0.3703 | −0.0123 |
| C | −0.2048 | 0.3517 | −0.0071 |
| C | −1.3922 | −0.4115 | 0.0027 |
| C | −0.2755 | 1.7359 | −0.0103 |
| C | −2.6218 | 0.2289 | 0.0090 |
| C | −1.5208 | 2.3738 | −0.0036 |
| H | 0.6295 | 2.3289 | −0.0182 |
| C | −2.6840 | 1.6266 | 0.0060 |
| H | −3.5367 | −0.3485 | 0.0162 |
| H | −1.5612 | 3.4560 | −0.0060 |
| H | −3.6509 | 2.1144 | 0.0115 |
| O | −1.2179 | −1.7632 | 0.0053 |
| C | −2.3947 | −2.5632 | 0.0086 |
| H | −2.9975 | −2.3741 | −0.8834 |
| H | −2.0539 | −3.5958 | 0.0072 |
| H | −2.9922 | −2.3750 | 0.9045 |
| O | 4.5559 | −0.0196 | −0.0561 |
| H | 4.6673 | 0.4528 | 0.7762 |
| H | 2.2436 | 0.9927 | 0.8697 |

TABLE S8
Ion SO₃—CH₃⁻ coordinates (Ångström)

| Atoms | X | Y | Z |
|---|---|---|---|
| S | 0.4871 | −0.0419 | 0.0000 |
| O | 0.4625 | −0.8470 | 1.2266 |
| O | 0.4615 | −0.8520 | −1.2232 |
| O | −0.9029 | 0.8045 | −0.0011 |
| O | 1.4865 | 1.0246 | −0.0024 |
| C | −2.1045 | 0.0177 | 0.0001 |
| H | −2.1509 | −0.5999 | 0.8983 |
| H | −2.9268 | 0.7292 | −0.0029 |
| H | −2.1496 | −0.6052 | −0.8946 |

TABLE S9
Oxygen coordinates (Ångström)

| Atoms | X | Y | Z |
|---|---|---|---|
| O | 0.0000 | 0.0000 | 0.5933 |
| O | 0.0000 | 0.0000 | −0.5933 |

TABLE S10
Part2-radical2 (α,β-C—C cleavage-CH₃) coordinates (Ångström)

| Atoms | X | Y | Z |
|---|---|---|---|
| C | −1.8850 | −0.4220 | −0.0238 |
| H | −1.8410 | −1.0472 | −0.9193 |
| C | −3.1411 | 0.4269 | −0.0227 |
| H | −3.1399 | 1.0840 | 0.8521 |
| H | −3.1658 | 1.0449 | −0.9210 |
| O | −0.7828 | 0.4919 | −0.0129 |
| C | 0.4547 | 0.0033 | −0.0070 |
| C | 1.4988 | 1.0353 | 0.0071 |
| C | 0.7848 | −1.3468 | −0.0132 |
| C | 2.8663 | 0.5748 | 0.0140 |
| C | 2.1227 | −1.7189 | −0.0055 |
| H | 0.0133 | −2.1052 | −0.0247 |
| C | 3.1623 | −0.7568 | 0.0078 |
| H | 3.6373 | 1.3364 | 0.0251 |
| H | 2.3747 | −2.7722 | −0.0104 |
| H | 4.1924 | −1.0910 | 0.0134 |
| O | 1.1922 | 2.2451 | 0.0131 |
| O | −4.2921 | −0.4005 | −0.0521 |
| H | −4.3458 | −0.8595 | 0.7932 |
| H | −1.8458 | −1.0563 | 0.8670 |

TABLE S11
2,4-dimethoxy-bezoic acid coordinates (Ångström)

| Atoms | X | Y | Z |
|---|---|---|---|
| C | −1.3135 | −0.5039 | −0.1086 |
| C | −0.7130 | 0.7602 | −0.2774 |
| C | 0.6579 | 0.8946 | −0.2283 |
| C | 1.4633 | −0.2308 | −0.0172 |
| C | 0.8719 | −1.4780 | 0.1553 |
| C | −0.5091 | −1.6214 | 0.1123 |
| H | 1.0871 | 1.8797 | −0.3641 |
| H | 1.4977 | −2.3457 | 0.3234 |
| H | −0.9487 | −2.6006 | 0.2452 |
| O | −1.5099 | 1.8562 | −0.5173 |
| O | −2.6600 | −0.5278 | −0.1877 |
| C | −2.0956 | 2.3908 | 0.6772 |
| H | −1.3134 | 2.7529 | 1.3495 |
| H | −2.7287 | 3.2209 | 0.3694 |
| H | −2.6998 | 1.6357 | 1.1847 |
| C | 2.9386 | −0.1345 | 0.0329 |
| O | 3.6796 | −1.0761 | 0.2136 |
| C | −3.3090 | −1.7878 | −0.0187 |
| H | −4.3719 | −1.5891 | −0.1299 |
| H | −2.9830 | −2.4954 | −0.7843 |
| H | −3.1104 | −2.1933 | 0.9759 |
| O | 3.3974 | 1.1118 | −0.1397 |
| H | 4.3654 | 1.0885 | −0.0872 |

TABLE S12
Quinone coordinates (Ångström)

| Atoms | X | Y | Z |
|---|---|---|---|
| O | 1.7265 | 1.3523 | 0.0000 |
| C | 0.6612 | 0.7772 | 0.0000 |
| C | 0.6612 | −0.7772 | 0.0000 |
| C | −0.6372 | 1.4587 | 0.0000 |
| C | −0.6372 | −1.4587 | 0.0000 |
| C | −1.7653 | 0.7350 | 0.0000 |
| H | −0.6344 | 2.5419 | 0.0000 |
| C | −1.7653 | −0.7350 | 0.0000 |
| H | −0.6344 | −2.5419 | 0.0000 |
| H | −2.7293 | 1.2301 | 0.0000 |
| H | −2.7293 | −1.2301 | 0.0000 |
| O | 1.7265 | −1.3523 | 0.0000 |

TABLE S13
Ion SO₃—CH₂—CH₂—OH⁻ coordinates (Ångström)

| Atoms | X | Y | Z |
|---|---|---|---|
| S | −1.3768 | −0.0276 | 0.0000 |
| O | −1.4573 | −0.8303 | −1.2248 |
| O | −1.4571 | −0.8300 | 1.2251 |
| O | 0.1127 | 0.6336 | −0.0002 |
| O | −2.2262 | 1.1616 | −0.0001 |
| C | 1.2125 | −0.2888 | −0.0001 |
| H | 1.1716 | −0.9156 | −0.8941 |
| H | 1.1715 | −0.9156 | 0.8939 |
| C | 2.4702 | 0.5496 | 0.0000 |
| H | 2.4852 | 1.1861 | −0.8901 |
| H | 2.4851 | 1.1861 | 0.8902 |
| O | 3.5587 | −0.3620 | 0.0001 |
| H | 4.3722 | 0.1521 | −0.0001 |

TABLE S14
2-OH-acetylaldehyde coordinates (Ångström)

| Atoms | X | Y | Z |
|---|---|---|---|
| C | −0.7098 | 0.3568 | −0.0854 |
| H | −0.5101 | 1.4226 | −0.3034 |
| C | 0.5083 | −0.5291 | −0.0338 |
| H | 0.4967 | −1.1455 | −0.9374 |
| O | 1.7084 | 0.2167 | −0.0415 |
| H | 1.7878 | 0.6585 | 0.8108 |
| H | 0.4280 | −1.1972 | 0.8297 |
| O | −1.8326 | −0.0547 | 0.0810 |

TABLE S15
Intermediate 1 coordinates (Ångström)

| Atoms | X | Y | Z |
|---|---|---|---|
| C | 4.3462 | −0.1536 | −0.3456 |
| C | 3.5432 | −1.2450 | −0.7170 |
| C | 2.1719 | −1.1082 | −0.8001 |
| C | 1.5502 | 0.1099 | −0.5033 |
| C | 2.3473 | 1.1920 | −0.1597 |
| C | 3.7358 | 1.0668 | −0.0818 |
| H | 1.5898 | −1.9701 | −1.1127 |
| H | 1.9099 | 2.1585 | 0.0508 |
| H | 4.3264 | 1.9325 | 0.1867 |
| O | 4.1383 | −2.4509 | −1.0245 |
| O | 5.6851 | −0.3884 | −0.2918 |
| C | 4.4416 | −3.2239 | 0.1427 |
| H | 3.5206 | −3.4866 | 0.6711 |
| H | 4.9394 | −4.1288 | −0.2016 |
| H | 5.1053 | −2.6704 | 0.8111 |
| C | 0.0435 | 0.1635 | −0.6356 |
| H | −0.1945 | −0.2489 | −1.6190 |
| C | −0.7905 | −0.7062 | 0.3458 |
| C | −0.0353 | −1.4662 | 1.4370 |
| H | 0.6012 | −2.2220 | 0.9690 |
| O | −0.4600 | 1.5074 | −0.7896 |
| O | 0.7593 | −0.6483 | 2.2756 |
| H | 0.5035 | 0.2739 | 2.0953 |
| H | −0.7939 | −1.9955 | 2.0262 |
| S | −0.7521 | 2.5628 | 0.4495 |
| O | −2.2659 | 2.4228 | 1.0656 |
| H | −2.8737 | 2.9358 | 0.5097 |
| O | −1.5382 | 3.4526 | −0.8773 |
| O | 0.0086 | 3.8433 | 0.5608 |
| O | −0.1516 | 1.7413 | 1.5579 |
| H | −1.5511 | −0.0734 | 0.8011 |
| O | −1.4494 | −1.7325 | −0.4301 |
| C | −2.7976 | −1.8697 | −0.1807 |
| C | −3.6761 | −0.8607 | −0.6135 |
| C | −3.2829 | −2.9993 | 0.4518 |
| C | −5.0414 | −1.0007 | −0.3872 |
| C | −4.6546 | −3.1453 | 0.6659 |
| H | −2.5760 | −3.7573 | 0.7691 |
| C | −5.5220 | −2.1453 | 0.2522 |
| H | −5.7325 | −0.2343 | −0.7129 |
| H | −5.0327 | −4.0321 | 1.1588 |
| H | −6.5877 | −2.2457 | 0.4196 |
| O | −3.0895 | 0.1900 | −1.2381 |
| C | −3.9173 | 1.2893 | −1.5957 |
| H | −4.4322 | 1.6907 | −0.7185 |
| H | −3.2496 | 2.0417 | −2.0121 |
| H | −4.6522 | 0.9942 | −2.3492 |
| C | 6.5188 | 0.7067 | 0.0751 |
| H | 7.5349 | 0.3194 | 0.0792 |
| H | 6.4378 | 1.5166 | −0.6542 |
| H | 6.2618 | 1.0760 | 1.0710 |

TABLE S16
Intermediate 2 coordinates (Ångström)

| Atoms | X | Y | Z |
|---|---|---|---|
| C | 4.3135 | −0.3583 | −0.4198 |
| C | 3.4104 | −1.4021 | −0.6805 |
| C | 2.0497 | −1.1605 | −0.7045 |
| C | 1.5504 | 0.1202 | −0.4604 |
| C | 2.4428 | 1.1519 | −0.2014 |
| C | 3.8184 | 0.9214 | −0.1867 |
| H | 1.3793 | −1.9888 | −0.9142 |
| H | 2.0754 | 2.1525 | −0.0086 |
| H | 4.4936 | 1.7439 | 0.0087 |
| O | 3.8922 | −2.6694 | −0.9362 |
| O | 5.6322 | −0.6948 | −0.4346 |
| C | 4.2275 | −3.3793 | 0.2621 |
| H | 3.3458 | −3.4738 | 0.9020 |
| H | 4.5688 | −4.3667 | −0.0439 |
| H | 5.0248 | −2.8675 | 0.8060 |
| C | 0.0594 | 0.3113 | −0.4083 |
| H | −0.4373 | −0.4060 | −1.0599 |
| C | −0.5560 | 0.2604 | 0.9981 |
| C | 0.0150 | −0.7831 | 1.9490 |
| H | 0.0937 | −1.7480 | 1.4409 |
| O | −0.3148 | 1.6063 | −0.8874 |
| O | 1.3029 | −0.4273 | 2.4313 |
| H | 1.3133 | 0.5390 | 2.4537 |
| H | −0.6811 | −0.8924 | 2.7878 |
| S | −0.7442 | 2.8311 | 0.1665 |
| O | −2.2040 | 2.6799 | 0.9265 |
| H | −2.8131 | 3.2039 | 0.3822 |
| O | −1.2794 | 3.6617 | −0.9706 |
| O | 0.2786 | 3.6854 | 0.8053 |
| O | −0.3198 | 1.5377 | 1.4974 |
| H | −1.6325 | 0.0674 | 0.8852 |
| O | −1.7845 | −2.3895 | −0.4671 |
| C | −2.9999 | −2.2129 | −0.2377 |
| C | −3.6813 | −0.9951 | −0.6981 |
| C | −3.8000 | −3.1732 | 0.4829 |
| C | −5.0369 | −0.8016 | −0.4477 |
| C | −5.1264 | −2.9560 | 0.7137 |
| H | −3.2949 | −4.0703 | 0.8217 |
| C | −5.7460 | −1.7697 | 0.2486 |
| H | −5.5417 | 0.0918 | −0.7894 |
| H | −5.7165 | −3.6859 | 1.2539 |
| H | −6.8004 | −1.6122 | 0.4400 |
| O | −2.8939 | −0.1434 | −1.3433 |
| C | −3.4566 | 1.0877 | −1.8099 |
| H | −3.8973 | 1.6409 | −0.9789 |
| H | −2.6290 | 1.6478 | −2.2356 |
| H | −4.2134 | 0.8869 | −2.5708 |
| C | 6.5704 | 0.3498 | −0.1978 |
| H | 7.5533 | −0.1101 | −0.2700 |
| H | 6.4782 | 1.1346 | −0.9528 |
| H | 6.4357 | 0.7769 | 0.7991 |

TABLE S17
Transition state 1-2 coordinates (Ångström)

| Atoms | X | Y | Z |
|---|---|---|---|
| C | 4.2442 | −0.2545 | −0.4411 |
| C | 3.3699 | −1.3198 | −0.7189 |
| C | 2.0090 | −1.1038 | −0.8050 |
| C | 1.4782 | 0.1722 | −0.6006 |
| C | 2.3390 | 1.2246 | −0.3311 |
| C | 3.7177 | 1.0200 | −0.2592 |
| H | 1.3623 | −1.9480 | −1.0246 |
| H | 1.9573 | 2.2267 | −0.1786 |
| H | 4.3676 | 1.8604 | −0.0548 |
| O | 3.8813 | −2.5838 | −0.9210 |
| O | 5.5654 | −0.5699 | −0.3876 |
| C | 4.1630 | −3.2613 | 0.3100 |
| H | 3.2540 | −3.3374 | 0.9135 |
| H | 4.5144 | −4.2571 | 0.0459 |
| H | 4.9373 | −2.7355 | 0.8734 |
| C | −0.0226 | 0.3128 | −0.6192 |
| H | −0.4410 | −0.2326 | −1.4594 |
| C | −0.7054 | −0.1899 | 0.6483 |
| C | 0.0005 | −1.0105 | 1.7275 |
| H | 0.3429 | −1.9425 | 1.2852 |
| O | −0.4274 | 1.6734 | −0.8591 |
| O | 1.1383 | −0.4012 | 2.3106 |
| H | 0.9372 | 0.5390 | 2.3992 |
| H | −0.7512 | −1.2478 | 2.4876 |
| S | −0.7971 | 2.6000 | 0.4418 |
| O | −2.3489 | 2.4554 | 0.8922 |
| H | −2.8968 | 3.0692 | 0.3757 |
| O | −1.2988 | 3.7211 | −0.7543 |
| O | 0.0300 | 3.7248 | 0.9427 |
| O | −0.3645 | 1.4922 | 1.4509 |
| H | −1.7563 | 0.0177 | 0.7628 |
| O | −1.3671 | −1.8801 | −0.2081 |
| C | −2.6805 | −1.9764 | −0.1059 |
| C | −3.5342 | −0.9925 | −0.6850 |
| C | −3.3014 | −3.0258 | 0.5864 |
| C | −4.9150 | −1.0802 | −0.5693 |
| C | −4.6888 | −3.1206 | 0.6931 |
| H | −2.6579 | −3.7767 | 1.0332 |
| C | −5.4968 | −2.1482 | 0.1226 |
| H | −5.5502 | −0.3268 | −1.0180 |
| H | −5.1312 | −3.9516 | 1.2307 |
| H | −6.5755 | −2.2062 | 0.2042 |
| O | −2.8821 | 0.0092 | −1.3544 |
| C | −3.6563 | 1.1083 | −1.8077 |
| H | −4.2137 | 1.5630 | −0.9834 |
| H | −2.9478 | 1.8298 | −2.2136 |
| H | −4.3534 | 0.8039 | −2.5937 |
| C | 6.4766 | 0.4925 | −0.1237 |
| H | 7.4679 | 0.0458 | −0.1367 |
| H | 6.4107 | 1.2622 | −0.8968 |
| H | 6.2852 | 0.9350 | 0.8572 |

TABLE S18
Intermediate 2′ coordinates (Ångström)

| Atoms | X | Y | Z |
|---|---|---|---|
| C | −2.8111 | −0.8975 | −0.0521 |
| C | −2.6885 | 0.2753 | −0.8147 |
| C | −1.4404 | 0.7731 | −1.1384 |
| C | −0.2830 | 0.1261 | −0.7032 |
| C | −0.4033 | −1.0388 | 0.0425 |
| C | −1.6573 | −1.5557 | 0.3649 |
| H | −1.3847 | 1.6833 | −1.7275 |
| H | 0.4840 | −1.5534 | 0.3915 |
| H | −1.7226 | −2.4665 | 0.9451 |
| O | −3.8264 | 0.9251 | −1.2477 |
| O | −4.0822 | −1.3061 | 0.2111 |
| C | −4.3736 | 1.7861 | −0.2423 |
| H | −3.6393 | 2.5449 | 0.0430 |
| H | −5.2473 | 2.2663 | −0.6796 |
| H | −4.6713 | 1.2122 | 0.6387 |
| C | 1.0532 | 0.7643 | −0.9644 |
| H | 1.0151 | 1.3680 | −1.8726 |
| C | 1.6185 | 1.5875 | 0.2039 |
| C | 0.6536 | 2.4862 | 0.9504 |
| H | 0.1247 | 3.1050 | 0.2144 |
| O | 2.0589 | −0.2360 | −1.1821 |
| O | −0.2540 | 1.7291 | 1.7386 |
| H | −0.7857 | 2.3566 | 2.2386 |
| H | 1.2439 | 3.1479 | 1.5920 |
| S | 3.1835 | −0.5083 | 0.0308 |
| O | 4.3468 | 0.4007 | 0.0672 |
| O | 3.9267 | −1.5281 | −1.1381 |
| O | 2.9276 | −1.5970 | 0.9994 |
| O | 2.1743 | 0.6093 | 1.0360 |
| H | 2.4026 | 2.2410 | −0.2068 |
| C | −4.2334 | −2.4910 | 0.9865 |
| H | −5.3050 | −2.6467 | 1.0875 |
| H | −3.7848 | −3.3482 | 0.4781 |
| H | −3.7852 | −2.3692 | 1.9759 |
| H | 3.3369 | −2.2788 | −1.2842 |

TABLE S19
Transition state 2′-3 coordinates (Ångström)

| Atoms | X | Y | Z |
|---|---|---|---|
| C | −2.7790 | −1.0346 | −0.0341 |
| C | −2.7511 | 0.1862 | −0.7266 |
| C | −1.5455 | 0.7769 | −1.0571 |
| C | −0.3349 | 0.1731 | −0.7057 |
| C | −0.3633 | −1.0493 | −0.0412 |
| C | −1.5740 | −1.6524 | 0.2940 |
| H | −1.5664 | 1.7104 | −1.6136 |
| H | 0.5670 | −1.5321 | 0.2354 |
| H | −1.5679 | −2.5981 | 0.8195 |
| O | −3.9386 | 0.7938 | −1.0758 |
| O | −4.0135 | −1.5253 | 0.2562 |
| C | −4.4438 | 1.6268 | −0.0254 |
| H | −3.7178 | 2.4092 | 0.2156 |
| H | −5.3629 | 2.0782 | −0.3950 |
| H | −4.6585 | 1.0342 | 0.8676 |
| C | 0.9515 | 0.8699 | −0.9856 |
| H | 0.8655 | 1.8684 | −1.4271 |
| C | 1.5685 | 1.5840 | 0.6761 |
| H | 2.2244 | 2.3627 | 0.2510 |
| C | 0.3220 | 2.1451 | 1.3477 |
| H | 0.6647 | 2.5802 | 2.2908 |
| O | 1.9438 | 0.1606 | −1.4226 |
| O | −0.2778 | 3.1905 | 0.6044 |
| H | −0.9879 | 2.8200 | 0.0709 |
| H | −0.3799 | 1.3402 | 1.5809 |
| S | 3.3387 | −0.7324 | −0.1774 |
| O | 4.4940 | −0.1109 | 0.8297 |
| H | 3.9844 | 0.3817 | 1.4979 |
| O | 4.1850 | −1.0047 | −1.3600 |
| O | 2.8367 | −1.9754 | 0.4486 |
| O | 2.1391 | 0.6062 | 1.2743 |
| C | −4.0702 | −2.7395 | 0.9989 |
| H | −5.1273 | −2.9508 | 1.1425 |
| H | −3.6036 | −3.5581 | 0.4455 |
| H | −3.5842 | −2.6229 | 1.9709 |

TABLE S20
Product 3 coordinates (Ångström)

| Atoms | X | Y | Z |
|---|---|---|---|
| C | 0.7910 | 1.0856 | −0.8828 |
| C | 2.0935 | 1.4314 | −0.4722 |
| C | 3.0839 | 0.4729 | −0.4237 |
| C | 2.7989 | −0.8493 | −0.7838 |
| C | 1.5093 | −1.1930 | −1.1922 |
| C | 0.5052 | −0.2361 | −1.2433 |
| H | 4.0792 | 0.7587 | −0.0977 |
| H | 1.2926 | −2.2198 | −1.4657 |
| H | −0.4946 | −0.5218 | −1.5436 |
| O | 2.3526 | 2.7319 | −0.1084 |
| O | −0.1069 | 2.0869 | −0.8784 |
| C | 1.9750 | 2.9971 | 1.2507 |
| H | 2.6119 | 2.4270 | 1.9326 |
| H | 2.1225 | 4.0633 | 1.4132 |
| H | 0.9269 | 2.7368 | 1.4198 |
| C | 3.8585 | −1.8647 | −0.6933 |
| H | 4.8512 | −1.4857 | −0.3961 |
| C | −0.5600 | −0.4470 | 1.6833 |
| H | −1.0730 | −1.3313 | 1.2660 |
| C | 0.8086 | −0.6837 | 2.2635 |
| H | 0.7235 | −0.5353 | 3.3444 |
| O | 3.6886 | −3.0476 | −0.9120 |
| O | 1.2649 | −2.0029 | 2.0457 |
| H | 1.5006 | −2.0878 | 1.1147 |
| H | 1.5032 | 0.0718 | 1.8783 |
| S | −4.0525 | −0.8997 | −0.3131 |
| O | −3.7821 | −0.2317 | 1.2111 |
| H | −2.9138 | 0.2198 | 1.2268 |
| O | −4.3167 | 0.3090 | −1.1659 |
| O | −2.6922 | −1.4591 | −0.6461 |
| O | −1.1009 | 0.6389 | 1.6944 |
| C | −1.4367 | 1.7772 | −1.3032 |
| H | −2.0053 | 2.6968 | −1.1885 |
| H | −1.4402 | 1.4654 | −2.3509 |
| H | −1.8670 | 0.9924 | −0.6852 |

### REFERENCES

[1] W. Boerjan, J. Ralph, M. Baucher, Annu. Rev. Plant Biol. 2003, DOI: 10.1146/annurev.arplant.54.031902.134938.
[2] FAO, “FAOSTAT Forestry Production and Trade,” 2018.
[3] S. Sen, H. Sadeghifar, D. S. Argyropoulos, Biomacromolecules 2013, DOI 10.1021/bm4010172.
[4] J. J. Meister, in Chem. Modif. Lignocellul. Mater., 2017.
[5] N. Chen, L. A. Dempere, Z. Tong, ACS Sustain. Chem. Eng. 2016, DOI 10.1021/acssuschemeng.6b01209.
[6] S. Jairam, Z. Tong, L. Wang, B. Welt, ACS Sustain. Chem. Eng. 2013, DOI 10.1021/sc4003196.
[7] A. J. Ragauskas, G. T. Beckham, M. J. Biddy, R. Chandra, F. Chen, M. F. Davis, B. H. Davison, R. a Dixon, P. Gilna, M. Keller, et al., Science 2014, DOI 10.1126/science.1246843.
[8] H. Kim, J. Ralph, Org. Biomol. Chem. 2010, DOI: 10.1039/b916070a.
[9] H. Wang, B. yang, Q. Zhang, W. Zhu, Renew. Sust. Energ. Rev. 2020, DOI:10.1016/j.rser.2019.109612.
[10] H. Liu, H. Li, N. Luo, F. Wang, Acs. Catal. 2020, DOI 10.1021/acscatal.9b03768.
[11] Z. Sun, B. Fridrich, A. De Santi, S. Elangovan, K. Barta, Chem. Rev. 2018, DOI 10.1021/acs.chemrev.7b00588.
[12] J. Kibet, L. Khachatryan, B. Dellinger, Environ. Sci. Technol. 2012, DOI 10.1021/es302942c.
[13] Y. Ye, J. Fan, J. Chang, J. Anal. Appl. Pyrolysis 2012, DOI 10.1016/j.jaap.2011.12.005.
[14] M. Dawange, M. V. Galkin, J. S. M. Samec, ChemCatChem 2015, DOI 10.1002/cctc.201402825.
[15] M. V. Galkin, S. Sawadjoon, V. Rohde, M. Dawange, J. S. M. Samec, ChemCatChem 2014, DOI 10.1002/cctc.201300540.
[16] G. T. Beckham, C. W. Johnson, E. M. Karp, D. Salvachúa, D. R. Vardon, Curr. Opin. Biotechnol. 2016, DOI 10.1016/j.copbio.2016.02.030.
[17] L. Petitjean, R. Gagne, E. Beach, D. Xiao, P. T. Anastas, Green. Chem. 2016, DOI: 10.1039/C5GC01464F.
[18] J. Xin, P. Zhang, M. P. Wolcott, X. Zhang, J. Zhang, Bioresour. Technol. 2014, DOI 10.1016/j.biortech.2013.12.092.
[19] C. Zhu, W. Ding, T. Shen, C. Tang, C. Sun, S. Xu, Y. Chen, J. Wu, H. Ying, ChemSusChem 2015, DOI 10.1002/cssc.201500048.
[20] R. Ma, M. Guo, X. Zhang, ChemSusChem 2014, DOI 10.1002/cssc.201300964.
[21] J. Long, Y. Xu, T. Wang, R. Shu, Q. Zhang, X. Zhang, J. Fu, L. Ma, BioResources 2014 DOI 10.15376/biores.9.4.7162-7175.
[22] G. Fang, J. Gao, D. D. Dionysiou, C. Liu, D. Zhou, Environ. Sci. Technol. 2013, DOI 10.1021/es400262n.
[23] C. S. Lancefield, O. S. Ojo, F. Tran, N. J. Westwood, Angew. Chemie—Int. Ed. 2015, DOI 10.1002/anie.201409408.
[24] T. H. Parsell, B. C. Owen, I. Klein, T. M. Jarrell, C. L. Marcum, L. J. Haupert, L. M. Amundson, H. I. Kenttämaa, F. Ribeiro, J. T. Miller, et al., Chem. Sci. 2013, DOI 10.1039/C2SC21657D
[25] T. Li, H. Lin, X. Ouyang, X. Qiu, Z. Wan, ACS Catal. 2019, DOI 10.1021/acscatal.9b01452.
[26] L. Shuai, M. T. Amiri, Y. M. Questell-Santiago, F. Héroguel, Y. Li, H. Kim, R. Meilan, C. Chapple, J. Ralph, J. S. Luterbacher, Science (80-.). 2016, DOI 10.1126/science.aaf7810.
[27] W. Lan, M. T. Amiri, C. M. Hunston, J. S. Luterbacher, Angew. Chemie—Int. Ed. 2018, DOI 10.1002/anie.201710838.
[28] K. H. Kim, T. Dutta, E. D. Walter, N. G. Isern, J. R. Cort, B. A. Simmons, S. Singh, ACS Sustain. Chem. Eng. 2017, DOI 10.1021/acssuschemeng.6b03102.
[29] P. J. Deuss, M. Scott, F. Tran, N. J. Westwood, J. G. De Vries, K. Barta, J. Am. Chem. Soc. 2015, DOI 10.1021/jacs.5b03693.
[30] A. G. Sergeev, J. F. Hartwig, Science 2011, DOI 10.1126/science.1200437.
[31] F. Gu, P. Posoknistakul, S. Shimizu, T. Yokoyama, Y. Jin, Y. Matsumoto, J. Wood Sci. 2014, DOI 10.1007/s10086-014-1411-5.
[32] T. Kleine, J. Buendia, C. Bolm, Green Chem. 2013, DOI 10.1039/c2gc36456e.
[33] S. Dabral, J. Mottweiler, T. Rinesch, C. U. Bolm, Green Chem. 2015, DOI 10.1039/c5gc00186b.
[34] J. D. Nguyen, B. S. Matsuura, C. R. J. Stephenson, J. Am. Chem. Soc. 2014, DOI 10.1021/ja4113462.
[35] X. Huang, T. I. Korenyi, M. D. Boot, E. J. M. Hensen, ChemSusChem 2014, DOI 10.1002/cssc.201402094.
[36] M. V. Galkin, J. S. M. Samec, ChemSusChem 2016, DOI 10.1002/cssc.201600237.
[37] J. Zakzeski, P. C. A. Bruijnincx, A. L. Jongerius, B. M. Weckhuysen, Chem. Rev. 2010, DOI 10.1021/cr900354u.
[38] S. H. Lim, K. Nahm, C. S. Ra, D. W. Cho, U. C. Yoon, J. A. Latham, D. Dunaway-Mariano, P. S. Mariano, J. Org. Chem. 2013, DOI 10.1021/jo401680z.
[39] R. Zhu, B. Wang, M. Cui, J. Deng, X. Li, Y. Ma, Y. Fu, Green Chem. 2016, DOI 10.1039/c5gc02347e.
[40] A. Rahimi, A. Azarpira, H. Kim, J. Ralph, S. S. Stahl, J. Am. Chem. Soc. 2013, DOI 10.1021/ja401793n.
[41] A. Das, A. Rahimi, A. Ulbrich, M. Alherech, A. H. Motagamwala, A. Bhalla, L. Da Costa Sousa, V. Balan, J. A. Dumesic, E. L. Hegg, et al., ACS Sustain. Chem. Eng. 2018, DOI 10.1021/acssuschemeng.7b03541.
[42] C. Zhang, H. Li, J. Lu, X. Zhang, K. E. Macarthur, M. Heggen, F. Wang, ACS Catal. 2017, DOI 10.1021/acscatal.7b00148.
[43] I. Bosque, G. Magallanes, M. Rigoulet, M. D. Karkss, C. R. J. Stephenson, ACS Cent. Sci. 2017, DOI 10.1021/acscentsci.7b00140.
[44] S. K. Hanson, R. Wu, L. a P. Silks, Angew. Chem. Int. Ed. Engl. 2012, DOI 10.1002/anie.201107020.
[45] L. J. Mitchell, C. J. Moody, J. Org. Chem. 2014, DOI 10.1021/jo5020917.
[46] J. Mottweiler, M. Puche, C. Rauber, T. Schmidt, P. Concepción, A. Corma, C. Bolm, ChemSusChem 2015, DOI 10.1002/cssc.201500131.
[47] G. F. De Gregorio, R. Prado, C. Vriamont, X. Erdocia, J. Labidi, J. P. Hallett, T. Welton, ACS Sustain. Chem. Eng. 2016, DOI 10.1021/acssuschemeng.6b01339.
[48] J. Duan, D. L. Kasper, Glycobiology 2011, DOI 10.1093/glycob/cwq171.
[49] W. J. Sagues, H. Bao, J. L. Nemenyi, Z. Tong, ACS Sustain. Chem. Eng. 2018, DOI 10.1021/acssuschemeng.7b04500.
[50] P. Neta, R. E. Huie, A. B. Ross, J. Phys. Chem. Ref. Data 1988, DOI 10.1063/1.555808.
[51] P. Wardman, J. Phys. Chem. Ref. Data 1989, DOI 10.1063/1.555843.
[52] J. Zeng, C. G. Yoo, F. Wang, X. Pan, W. Vermerris, Z. Tong, ChemSusChem 2015, DOI 10.1002/cssc.201403128.
[53] G. P. Anipsitakis, D. D. Dionysiou, Environ. Sci. Technol. 2003, DOI 10.1021/es0263792.
[54] P. Spannring, V. Yazerski, P. C. A. Bruijnincx, B. M. Weckhuysen, R. J. M. Klein Gebbink, Chem.—A Eur. J. 2013, DOI 10.1002/chem.201301371.
[55] K. Okuda, M. Umetsu, S. Takami, T. Adschiri, in Fuel Process. Technol., 2004. DOI. 10.1016/j.fuproc.2003.11.027
[56] M. Saisu, T. Sato, M. Watanabe, T. Adschiri, K. Arai, Energy and Fuels 2003, DOI 10.1021/ef0202844.
[57] Z. Yuan, S. Cheng, M. Leitch, C. C. Xu, Bioresour. Technol. 2010, DOI 10.1016/j.biortech.2010.06.140.
[58] J. Buendia, J. Mottweiler, C. Bolm, Chem.—A Eur. J. 2011, DOI 10.1002/chem.201101579.
[59] G. P. F. van Strijdonck, J. A. E. H. van Haare, P. J. M. Hönen, R. C. G. M. van den Schoor, M. C. Feiters, J. G. M. van der Linden, J. J. Steggerda, R. J. M. Nolte, J. Chem. Soc. Dalt. Trans. 1997, DOI 10.1039/a602587k.
[60] Y. Zhao, D. G. Truhlar, Acc. Chem. Res. 2008, DOI 10.1021/ar700111a.
[61] S. Kim, S. C. Chmely, M. R. Nimlos, Y. J. Bomble, T. D. Foust, R. S. Paton, G. T. Beckham, J. Phys. Chem. Lett. 2011, DOI 10.1021/jz201182w.
[62] A. V. Marenich, C. J. Cramer, D. G. Truhlar, J. Phys. Chem. B 2009, DOI 10.1021/jp810292n.
[63] S. L. C. Ferreira, R. E. Bruns, H. S. Ferreira, G. D. Matos, J. M. David, G. C. Brandão, E.
G. P. da Silva, L. A. Portugal, P. S. dos Reis, A. S. Souza, Anal. Chim. Acta 2007, DOI 10.1016/j.aca.2007.07.011.
[64] G. E. P. Box, K. B. Wilson, J. R. Stat. Soc. B 1951, DOI 10.1111/j.2517-6161.1951.tb00067.x.
[65] M. J. G. Frisch, W. Trucks, H. B. Schlegel, G. E. Scuseria, M. A. Robb, J. R. Cheeseman, G. Scalmani, V. Barone, B. Mennucci, G. A. Petersson, Inc. Wallingford, CT 2016, DOI 111.

### Example 2

### 1. Experimental Section

### 1.1 Materials

In this study, sodium persulfate (>99.0%) was purchased from Sigma-Aldrich Inc. (USA). Ferrous sulfate heptahydrate (>99.0%) was purchased from ThermoFisher Scientific Inc. (USA). Municipal solid waste (MSW) was kindly provided by Synergy Burcell Technologies (GA, USA).

### 1.2 General Procedures for the Catalytical Oxidation of MSW

The MSW sample was used for the free radical persulfate oxidation reaction. In a typical run, 1.5 g MSW sample was dissolved in a 15 g ethanol (50 wt. %) and then loaded into a seal tube. 2.0 equivalent (EQ) of persulfate (weight based) and 5% molar fraction of Ferrous catalyst was then added in the tube and the reaction temperature was increased to the designed reaction temperature (>80° C.) to initiate the reaction. The reaction was kept in the seal tube for 24 hours. The product underwent a liquid/liquid extraction three times using ethyl acetate (EtOAc) and was then washed with water and the saturated sodium chloride solution. The organic oil was evaporated in a rotary evaporator to remove the solvent. The remainder in the tube after extraction was washed by water. All the liquid was collected and filtered through a weighted filter paper to collect insoluble solid residue. Both the dried oil product and solid residue were weighted to calculate the conversion yield and the solid residue yield.

### 1.3 General Procedures for the Gas Chromatography-Mass Spectrometry (GC-MS):

The gas chromatography-mass spectrometry (GC-MS, GC-Agilent 7820 and MS-Agilent 5977) was used to determine the chemical composition of the organic oil product. Helium was used as the carrier gas at a flow rate of 1 mL/min with 1:20 split ratio. In a typical run, 10 mg of the oil product was dissolved in 1 mL of organic solvents, dried with magnesium sulfate anhydrous and filtered with 0.22 μm syringe filter. An Agilent HP-5MS 5% Phenyl Methyl Silox 30 m×250 μm×0.25 μm column was set in the GC oven to separate the sample and the oven temperature ramped up from 50° C. to 280° C. Mass detector was set at 70 eV to generate comparable MS spectra with online database.

### 1.4 General Procedures for the Gel Permeation Chromatography (GPC):

The gel permeation chromatography was used to analyze the molar mass distribution of the depolymerized oil product. Three individual pore size organic GPC columns, Agilent PLgel Sum 10,000 A, Agilent PLgel 5 μm 100 A and Agilent PLgel 10 μm Mixed-B were connected in series and carried by a Agilent 1260 HPLC system. 5 mg of the oil product was dissolved in 1 mL HPLC grade tetrahydrofuran (THF), dried with magnesium sulfate anhydrous and filtered with 0.22 μm syringe filter. The sample was then injected into the column for analysis.

### 2. Results and Discussion

Our previous research has demonstrated that oxidizer persulfate could effectively depolymerize lignin dimer and industrial lignin into monophenolic aromatics under mild reaction conditions. In this study, we propose that the oxidizer persulfate with organic solvent could effectively depolymerize lignin and extract the liquid oil from the MSW, leaving the remaining unreacted carbohydrate for monomer sugar production. According to our previous study on the lignin dimer and industrial lignin, we chosen the optimized catalyst loading (2.0 EQ persulfate based on weight in combination with 5% ferrous) and kept the reaction time for 24 hrs. Considering the recalcitrance of the MSW after multiple-time pulping and recycling processes, the reaction temperature was increased from and 80° C., 120° C. to 140° C. The ethanol/water solution (50/50 wt. ratio) was applied to improve the solubility of the lignin. The THF/water (50/50) was also used as the solvent as the comparison.

FIG. 2.1 displays the yields of the depolymerized products from the MSW. When the temperature kept at 140° C., the depolymerization of MSW yielded 27.10% of the oil product, 33.53% of solid residue, and 39.37% water solubles. With the increase of reaction temperature, the liquid oil yield was increased from 11.91 wt. % to 27.10 wt. % (based on the MSW dried weight) and water-soluble part was also significantly increased from 8.34 wt. % to 39.37 wt. %. The increase of organic liquid indicated the more depolymerized products from the hydrophobic parts (lignin) or more extracted oil at a higher temperature. The change of reaction temperature also significantly reduced the production of solid residues. The increase of water-soluble parts and the decrease of the solid residues indicated that higher temperature might result in the degradation of carbohydrates.

FIG. 2.2 shows the GC-MS chromatogram of the depolymerized products from the MSW. It was observed that the yield of products derived from syringyl residues was higher than the yield of products derived from guaiacyl and p-hydroxyphenyl residues, which was due to a high ratio of S unit in the MSW. Also, a significant amount of fatty acid-based ester products might come from food wastes in the MSW, which was different from other pretreatment results only using biomass as raw materials.

TABLE 2.1
The compositions of solid residues after fractionation
using persulfate biomimetic catalyst

| Samples | Carbohydrates | lignin | unknown |
|---|---|---|---|
| Burcell Samples | 53.50% | 35.00% | 11.50% |
| 80° C. | 65.66% | 31.22% | 3.12% |
| 120° C. | 71.96% | 24.27% | 3.77% |
| 140° C. | 79.73% | 17.24% | 3.03% |

Table 2.1 shows the composition of the solid residues after fractionation at different reaction temperatures. In comparison with the original sample, the depolymerization process significantly increased carbohydrate compositions and the half of lignin has been removed from the MSA after depolymerization. At the 140° C. reaction temperature, half of lignin was removed and the carbohydrates reached 79.73% which could be easier to convert to monomer sugars by cellulase enzyme hydrolyzation.

TABLE 2.2
GPC results of the depolymerized products of Burcell MSW samples

| Samples | Mn(g/mol) | Mw(g/mol) | PDI |
|---|---|---|---|
| 120° C. (Ethanol/Water) | 427 | 2109 | 4.95 |
| 140° C. (Ethanol/Water) | 840 | 4902 | 5.83 |
| 140° C. (THF/Water) | 664 | 1488 | 2.24 |

The weight-average molecular weight (Mw), number-average molecular weight (Mn), and polydispersity index (PDI) are listed in Table 2.2. The Mw of organic oil for 140° C. was almost doubled than that for 120° C. in ethanol/water solution, which is reasonable since higher temperature usually leads to the re-condensation of the depolymerized lignin. However, at the same temperature (140° C.), the use of solvent (THF/water) could significantly decrease the molecular weight of the depolymerized lignin. It was observed that MSW had a better solubility in THF/water solvent than in ethanol/water solvent, that may lead to more effective depolymerization of lignin in MSW.

### 3. Conclusion

Our previous work shows that biomimetic persulfate catalyst could effectively depolymerize industrial lignin to monophenolic monomers. This study further demonstrates the effectiveness of the use of persulfate biomimetic catalyst as a lignin-first fractionation method to convert lignin in MSW into low molecular weight aromatics. The results show that the significant increase of carbohydrates in solid residues after this process from 53.50% to 79.73%. The benefits include 1) avoiding the use of complicated and harsh pretreatment process; 2) decreasing the reactor volume for the saccharification and fermentation process of carbohydrates; 3) reducing the dosage of hydrophobic enzyme for saccharification; 4) adding value from the lignin-based aromatics.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

1. A method for depolymerizing lignin comprising:
providing a first composition comprising a lignin and a catalyst composition, wherein the catalyst composition comprises a salt of S₂O₈²⁻ and a transition metal compound; and
depolymerizing at least a portion of the lignin to provide one or more of a low molecular weight aromatic monomer, an aliphatic hydrocarbon, or a combination thereof.

2. The method of claim 1, wherein the salt of S₂O₈²⁻ comprises Na₂S₂O₈, K₂S₂O₈ or a combination thereof.

3. The method of claim 1, wherein the transition metal compound is selected an iron transition compound.

4. The method of claim 3, wherein the iron transition compound is a ferrous sulfate heptohydrate.

5. The method of claim 1, wherein the depolymerizing is performed at a pressure of about 1 atmosphere.

6. The method of claim 1, wherein the depolymerizing is performed at a temperature about 70° C. to about 100° C.

7. The method of claim 1, wherein the depolymerizing time is about 12 hours to about 36 hours.

8. The method of claim 1, wherein the lignin is selected from the group consisting of agricultural lignin, wood lignin, lignin derived from municipal waste, Kraft lignin, organosolv lignin, and combinations thereof.

9. The method of claim 1, wherein the lignin is lignin derived from municipal waste.

10. The method of claim 1, wherein the first composition further includes a phenol.

11. The method of claim 1, wherein the depolymerization yield is up to about 76% of the lignin, wherein the depolymerizing is performed at a temperature about 70° C. to about 90° C.

12. The method of claim 1, wherein the product of the depolymerization is one or more of C8 to C20 aromatic compounds, C8 to C20 aliphatic compounds, or a combination thereof.

13. The method of claim 1, wherein depolymerizing does not include hydrogen peroxide.

14. The method of claim 1, wherein the catalyst composition does not include hydrogen peroxide.

15. A method for depolymerizing lignin comprising:
providing a first composition comprising a lignin and a catalyst composition, wherein the catalyst composition comprises a salt of S₂O₈²⁻ and a transition metal compound; and
depolymerizing at least a portion of the lignin at a temperature of about 130 to 140° C. to yield an oil product.

16. The method of claim 15, wherein the salt of S₂O₈²⁻ comprises Na₂S₂O₈, K₂S₂O₈ or a combination thereof.

17. The method of claim 15, wherein the transition metal compound is selected an iron transition compound, optionally wherein the iron transition compound is a ferrous sulfate heptohydrate.

18. (canceled)

19. The method of claim 15, wherein the depolymerizing is performed at a pressure of about 1 atmosphere, wherein the depolymerizing is performed at a temperature about 140° C., wherein the depolymerizing time is about 12 hours to about 36 hours.

20-21. (canceled)

22. The method of claim 15, wherein the lignin is selected from the group consisting of agricultural lignin, wood lignin, lignin derived from municipal waste, Kraft lignin, organosolv lignin, and combinations thereof optionally wherein the lignin is lignin derived from municipal waste.

23. (canceled)

24. The method of claim 15, wherein depolymerizing does not include hydrogen peroxide or wherein the catalyst composition does not include hydrogen peroxide.

25. (canceled)
